(19) United States
(12) Patent Application Publication
(10) Pub. No.: US 2026/0273859 A1
(43) Pub. Date: Sep. 17, 2026

(54) APPARATUS FOR PRODUCING THREE-DIMENSIONAL SCREEN-PRINTED WORKPIECES, AND METHOD THEREFOR

(71) Applicant: Exentis Knowledge GmbH, Stetten Ag (CH)

(72) Inventors: Michael Reichenbach, Waldkirch (DE); Uwe Bürkin, Emmendingen (DE)

(21) Appl. No.: 19/164,092

(22) PCT Filed: Mar. 3, 2024
(86) PCT No.: PCT/EP2024/055522

§ 371 Date: Sep. 10, 2025

(30) Foreign Application Priority Data
Mar. 11, 2023 (DE) 20 2023 101 180.2
Mar. 11, 2023 (EP) 23161357.1

(51) Int. Cl. B29C 64/379; B29C 64/245; B33Y 10/00; B33Y 30/00; B33Y 40/00

(52) U.S. Cl. CPC B29C 64/379; B29C 64/245; B33Y 10/00; B33Y 30/00; B33Y 40/00

(57) ABSTRACT

Apparatus for producing three-dimensional screen-printed workpieces, in particular a 3D screen-printing machine, having a printing device for the layerwise production of at least one screen-printed workpiece in a plurality of printing operations and having a transport device for the automated transport of at least one screen-printed workpiece and/or of a workpiece carrier towards and/or away from the printing device, wherein the transport device has a carrying section for the at least form-fitting reception of a workpiece carrier.

The present invention relates to an apparatus for producing three-dimensional screen-printed workpieces. The present invention likewise relates to a method for producing three-dimensional screen-printed workpieces.

A system for producing three-dimensional screen-printed workpieces is known from the prior art in document WO 2020/212371 A1 . In this system concept, a transport device formed as a belt conveyor is provided for the automated transport of at least one workpiece carrier. Workpiece carriers can advantageously be transported in an automated manner in the circuit between a printing device and at least one position spaced apart from the printing device by such a transport device. As a result, an overall high utilization of the printing device and therefore a high degree of productivity can be ensured.

The transport of a workpiece carrier by means of such a belt conveyor takes place with relatively large tolerances and a free positioning of the workpiece carrier on the respective belts. After the arrangement of a workpiece carrier within a printing device, a relatively complex fine positioning or fine alignment can consequently still be required in order to apply the respective printed layers of a screen-printed workpiece to one another with sufficient accuracy. The productivity of such a system can be limited due to the requirement of such a fine positioning or fine alignment.

In addition, due to the free positioning of the respective workpiece carriers on the belts, only a limited control over the exact position or during the transport of the workpiece carrier within the apparatus can be ensured. As a result, workpiece carriers can assume undesired positions or locations within the apparatus, as a result of which an increased manual handling or operating effort can be incurred.

Against the background set out above, the object of the present invention was to specify an apparatus for producing three-dimensional screen-printed workpieces which ensures an increased productivity as well as a transport of a workpiece carrier with improved operational reliability. The object likewise was to specify a method for producing three-dimensional screen-printed workpieces.

With regard to the apparatus, this object has been achieved by the subject matter of claim 1. A method according to the invention is subject of claim 50. Advantageous embodiments are subject of the dependent claims and are explained below.

According to the invention, an apparatus for producing three-dimensional screen-printed workpieces is provided. The apparatus is in particular a 3D screen printing machine, preferably an automated 3D screen-printing machine.

The apparatus according to the invention has a printing device for the layerwise production of at least one screen-printed workpiece in at least one printing operation or in a plurality of printing operations and a transport device for the automated transport of at least one screen-printed workpiece and/or of a workpiece carrier towards and/or away from the printing device. In this case, the transport device according to the invention has a carrying section for receiving a workpiece carrier in an at least form-fitting manner.

A screen-printed workpiece can accordingly be produced layerwise in at least one printing operation or in a plurality of printing operations. Before or after any printing operations for a screen-printed workpiece, the respective workpiece carrier can be moved by the transport device towards and/or away from the printing device. During operation, the printing device can therefore be loaded with different workpiece carriers, with the result that an overall high utilization of the printing device can be ensured. Downtimes of the printing device can be avoided or at least kept to a low degree as a result. Altogether, a high overall productivity of the system can be ensured as a result.

At the same time, by means of the configuration of the transport device with a carrying section for receiving a workpiece carrier in an at least form-fitting manner, a precise guidance of the respective workpiece carrier during transport by means of the transport device as well as an increased positioning accuracy can be ensured with reduced effort. Due to the form-fitting reception of a workpiece carrier, an overall high degree of transport reliability results. An undesired falling down or slipping down of the respective workpiece carrier from the conveying vehicle during the transport can be reliably avoided by such a configuration. In addition, a form-fitting reception of a workpiece carrier already ensures a high degree of positioning accuracy when a workpiece carrier is arranged within a printing device. A further fine positioning or fine alignment can be avoided or reduced at least to an extent.

In the present case, three-dimensional screen printing can be understood in a particularly preferred manner as an additive manufacturing method, in which a powder-based suspension is transferred with the aid of a doctor blade through a solid printing mask onto a substrate and dried. This procedure can be repeated several times until the desired component height or component shape in each case is achieved. In a final process step, the component produced in this way can be sintered. A screen-printed workpiece can be produced as a result.

In the present case, three-dimensional screen printing can likewise be understood in a particularly preferred manner as an additive manufacturing method, in which a powder-based suspension is transferred with the aid of a doctor blade through a solid printing mask onto a substrate and dried, wherein the desired component height or component shape in each case is already achieved by a single print. In a final process step, a component produced in this way can be sintered and a screen-printed workpiece can be produced. Insofar as a plurality of printing operations are mentioned in the present case, one printing operation can already be sufficient and suitable instead of this.

In the present case, a screen-printed workpiece can be understood in a preferred manner as a workpiece or a three-dimensional printed product which is to be subjected or has been subjected to a sintering step. This relates in particular to workpieces made of a metal, a ceramic, a glass material and/or a plastic material. In particular, alloys made of steel, nickel, copper, titanium and/or ceramic alloys come into consideration for this purpose.

Printed products made of plastic materials can be excluded or included by the designation “three-dimensional screen-printed workpiece”. In particular, there is also the possibility of subjecting printed workpiece layers made of plastic material to a sintering step.

In the present case, a screen-printed workpiece can likewise be understood as a workpiece or a three-dimensional printed product which has been produced without a sintering step or can be finished or is finished without a sintering step. Consequently, a final curing of printed layers can also take place without sintering steps. The curing of a screen-printed workpiece can advantageously also take place by UV curing and/or by a polymerization reaction and/or by drying, in particular by convection drying. Such a curing can in particular preferably be carried out when a final curing of printed layers is intended to take place without sintering steps.

A screen-printed workpiece within the meaning of the present invention can furthermore be a pharmaceutical product and/or a biological product. Such screen-printed workpieces can be produced inter alia from pharmaceutical powder materials and/or powder mixtures and/or granules and/or from biological materials. In particular, pharmaceutical products and/or biological products can be finished without sintering steps or can be sufficiently cured for the respective application.

Screen-printed workpieces produced from pharmaceutical powder materials and/or powder mixtures and/or granules can comprise medicaments, active sub-stances, auxiliaries, in particular fillers and/or binders and/or disintegrants and/or lubricants.

According to a preferred configuration of the present invention, the apparatus can be designed and/or configured for production under clean room conditions. In particular, the apparatus can be designed and/or configured for production under clean room conditions according to clean room classes A, B, C and/or D according to EU-GMP.

Further preferably, an apparatus according to the invention for producing screen-printed workpieces can be designed and/or configured for application in medical technology, in optical and/or laser technology, in aerospace technology, in semiconductor technology, in biotechnology and/or in medical and/or in pharmacological research.

Likewise, the apparatus according to the invention for producing screen-printed workpieces can be designed and/or configured for use and/or application as medical and/or pharmaceutical products, implants and/or sterile products and/or medicaments and/or for use and/or application as tablets for active substance ad-ministration.

According to a further preferred configuration, the transport device can be set up for the automated transport of at least one screen-printed workpiece and/or of a workpiece carrier towards and/or away from a printing table of the printing device. Such a printing table can have in particular a printing table plate.

A printing table or a printing table plate can advantageously be brought into contact with an underside of a workpiece carrier for carrying out a printing operation and support the latter during the printing operation. A printing table or a printing table plate therefore preferably does not serve for direct printing or as a direct printing base or for providing a surface to be printed. Rather, a printing table or a printing table plate can advantageously be designed and/or arranged for temporarily contacting, raising and/or supporting a workpiece carrier.

According to the present teaching, a workpiece carrier can further advantageously be provided and/or designed for direct printing. In this case, a workpiece carrier within the meaning of the present teaching can also have a coating on which printed layers can be applied. A workpiece carrier or a coating of such a workpiece carrier can therefore be provided as a direct printing base or for providing a surface to be printed and/or arranged or arrangeable within the apparatus.

According to the present invention, a screen-printed workpiece can be a workpiece which is built up on the workpiece carrier by three-dimensional screen printing in one or in a plurality of printing operations. In this case, the screen-printed workpiece is in particular a workpiece which, after completion of the printing operation and/or after completion of a sintering operation following the printing operation, can be released again from the workpiece carrier, in particular can be released non-destructively.

Between any printing operations for a screen-printed workpiece, the respective workpiece carrier can be released from the printing table or from the printing table plate or can be releasable therefrom. The individual layers of a screen-printed workpiece-in the case of a multilayer construction-can be dried between two successive printing operations in a position remote from the printing table or from the printing table plate.

In a further preferred manner, the transport device can be formed separately and/or independently of the printing device. As a result, an overall modular construction of the apparatus can be made possible or further promoted. The transport device can be configured independently of the printing device and can be arranged for connecting the printing device to at least one further device or position within the apparatus.

In addition, by means of a transport device formed separately and/or independently of the printing device, the supplementation and/or the replacement of the printing device can be simplified, in particular the supplementation with further units within the apparatus or the replacement of the printing device by another printing device.

Despite the transport device being formed separately and/or independently of the printing device, it can be adapted to the printing device, in particular in order to enable a reliable transport of a workpiece carrier and/or of a screen-printed workpiece towards and/or away from the printing device.

The transport device can particularly preferably run through the printing device. In this way, a particularly advantageous and space-saving transport of a workpiece carrier and/or of a screen-printed workpiece towards and/or away from the printing device can be ensured. The printing device can be connected particularly advantageously to further devices or apparatus modules as a result. In the case of an overall modular apparatus construction, a high degree of integration of the respective devices or apparatus modules can be ensured in this way.

According to a further preferred configuration, the transport device can have at least one transport rail and a conveying vehicle, arranged movably on the transport rail, for at least one screen-printed workpiece and/or at least one workpiece carrier. In this case, the carrying section for receiving a workpiece carrier can be arranged on the conveying vehicle.

By the configuration of the transport device with a transport rail and with a conveying vehicle arranged movably on the transport rail, relative movements of surfaces arranged in a contacting manner can be avoided or at least reduced. Operation-related particle abrasion can therefore be completely avoided or kept at a low level with such a configuration. Altogether, improved cleanliness conditions can therefore be provided for the individual production steps.

An undesired contamination of the screen-printed workpieces to be produced in each case on account of particles arising as a result of friction can be prevented with a transport device configured according to the invention. Consequently, screen-printed workpieces with high cleanliness or hygiene requirements can be produced with an apparatus according to the invention.

Finally, a preferred configuration of the transport device with at least one transport rail and a conveying vehicle arranged on the transport rail ensures a high degree of flexibility. The layout of the system can be adapted or newly or subsequently configured differently with only little effort. An adaptation of the apparatus layout can be carried out for example inline, via switches and/or bypasses. In this way, further devices can be integrated with only little effort in addition to the printing device or can be dismantled again from the apparatus, depending on the intended use and production requirement.

According to a further preferred configuration, the carrying section and/or the conveying vehicle, in particular the carrying section of the conveying vehicle and/or the carrying section of a superstructure of the conveying vehicle, can be designed for receiving and/or fixing a workpiece carrier in a form-fitting and/or force-fitting and/or material-bonded manner and/or at least in sections. A high degree of transport reliability results for the transport of a workpiece carrier by means of such a carrying section or conveying vehicle. An undesired falling down or slipping down of the respective workpiece carrier from the carrying section or conveying vehicle during the transport can be reliably avoided as a result.

According to a further preferred configuration, the carrying section and/or the conveying vehicle, in particular the carrying section of the conveying vehicle and/or the carrying section of a superstructure of the conveying vehicle, can be designed for receiving and/or fixing in a form-fitting and/or force-fitting and/or material-bonded manner and/or at least in sections a workpiece carrier with a side edge length or a plurality of side edge lengths of at least 50 mm, preferably of at least 100 mm, preferably of at least 150 mm, preferably of at least 200 mm, preferably of at least 250 mm, preferably of at least 300 mm, preferably of at least 350 mm, preferably of at least 400 mm, preferably of at least 450 mm, further preferably of at least 475 mm. The receiving and/or fixing and/or surrounding of a workpiece carrier dimensioned in this way enables the printing onto of a relatively large printing base or of a surface to be printed on with sufficient dimensions.

According to a still further preferred configuration, the carrying section and/or the conveying vehicle, in particular the carrying section of the conveying vehicle and/or the carrying section of a superstructure of the conveying vehicle, can be designed for receiving and/or fixing in a form-fitting and/or force-fitting and/or material-bonded manner and/or at least in sections a workpiece carrier with a side edge length or a plurality of side edge lengths of up to 1000 mm, preferably of up to 900 mm, preferably of up to 800 mm, preferably of up to 700 mm, preferably of up to 600 mm, preferably of up to 550 mm, further preferably of up to 525 mm, still further preferably of up to 500 mm. The receiving and/or fixing and/or surrounding of a workpiece carrier dimensioned in this way enables an overall compact and stable construction and good handling of the respective workpiece carriers during the positioning in or on the carrying section or the conveying vehicle and during the removal from or from the carrying section or conveying vehicle.

According to a still further preferred configuration, the carrying section and/or the conveying vehicle, in particular the carrying section of the conveying vehicle and/or the carrying section of a superstructure of the conveying vehicle, can be designed for receiving and/or fixing in a form-fitting and/or force-fitting and/or material-bonded manner and/or at least in sections a workpiece carrier with a side edge length or a plurality of side edge lengths of 50 mm to 1000 mm, preferably of 100 mm to 900 mm, preferably of 150 mm to 850 mm, preferably of 200 mm to 800 mm, preferably of 250 mm to 750 mm, preferably of 300 mm to 700 mm, preferably of 350 mm to 650 mm, preferably of 400 mm to 600 mm, preferably of 450 mm to 550 mm, further preferably of 475 to 525 mm. Such a dimensioning enables the provision of a sufficiently dimensioned printing base or of a sufficiently dimensioned surface to be printed with at the same time a compact and robust construction of the carrying section or of the conveying vehicle, in particular of the superstructure of the conveying vehicle.

In particular, the carrying section and/or the conveying vehicle, preferably the carrying section of the conveying vehicle and/or the carrying section of a superstructure of the conveying vehicle, can be designed for receiving and/or fixing a rectangular or square or substantially square workpiece carrier and/or at least in sections.

Additionally or alternatively, the carrying section and/or the conveying vehicle, in particular the carrying section of the conveying vehicle and/or the carrying section of a superstructure of the conveying vehicle, can be designed for receiving and/or enclosing a workpiece carrier in a defined and/or play-free manner. A surrounding of the workpiece carrier can be provided all around or completely around all side edges of the workpiece carrier or also in sections. The fixing reliability for a workpiece carrier can be further improved by such a configuration.

It can also be advantageous if the carrying section and/or the conveying vehicle, in particular the carrying section of the conveying vehicle and/or the carrying section of a superstructure of the conveying vehicle, has a frame device. Such a frame device can be, in particular, a frame device for receiving and/or fixing a workpiece carrier in a form-fitting and/or force-fitting and/or material-bonded manner and/or at least in sections. Such a frame device ensures a particularly secure receiving and/or fixing of a workpiece carrier with good accessibility of the workpiece carrier at the same time for the handling during the arrangement in and/or removal from the frame device.

In a further preferred configuration, a frame device can be designed for receiving and/or fixing a workpiece carrier with a side length or a plurality of side lengths of at least 50 mm, preferably of at least 100 mm, preferably of at least 150 mm, preferably of at least 200 mm, preferably of at least 250 mm, preferably of at least 300 mm, preferably of at least 350 mm, preferably of at least 400 mm, preferably of at least 450 mm, further preferably of at least 475 mm. The receiving and/or fixing and/or surrounding of a workpiece carrier dimensioned in this way enables the printing of a relatively large printing base or of a surface with sufficiently large dimensions. In a printing operation, numerous screen-printed workpieces can therefore be produced layerwise and a high level of economy can therefore be ensured.

In a still further preferred configuration, a frame device can be designed for receiving and/or fixing a workpiece carrier with a side length or a plurality of side lengths of up to 1000 mm, preferably of up to 900 mm, preferably of up to 800 mm, preferably of up to 700 mm, preferably of up to 600 mm, preferably of up to 550 mm, further preferably of up to 525 mm, still further preferably of up to 500 mm. The receiving and/or fixing and/or surrounding of a workpiece carrier dimensioned in this way enables an overall compact and stable construction of the frame device and also good handling of the respective workpiece carriers during the positioning within the respective frame device.

In a still further preferred configuration, a frame device can be designed for receiving and/or fixing a workpiece carrier with a side length or a plurality of side lengths of 50 mm to 1000 mm, preferably of 100 mm to 900 mm, preferably of 150 mm to 850 mm, preferably of 200 mm to 800 mm, preferably of 250 mm to 750 mm, preferably of 300 mm to 700 mm, preferably of 350 mm to 650 mm, preferably of 400 mm to 600 mm, preferably of 450 mm to 550 mm, further preferably of 475 to 525 mm. Such a dimensioning enables the provision of a sufficiently dimensioned printing base or of a surface to be printed with at the same time a compact and robust construction of the frame device.

Opposite inner edges of a frame device can in a further preferred manner have a distance from one another of at least 50 mm, preferably of at least 100 mm, preferably of at least 150 mm, preferably of at least 200 mm, preferably of at least 250 mm, preferably of at least 300 mm, preferably of at least 350 mm, preferably of at least 400 mm, preferably of at least 450 mm, further preferably of at least 475 mm. By means of such a configuration, a frame device can receive or surround workpiece carriers of sufficiently large dimensions.

In a further preferred manner, opposite inner edges of such a frame device can have a distance from one another of up to 1000 mm, preferably of up to 900 mm, preferably of up to 800 mm, preferably of up to 700 mm, preferably of up to 600 mm, preferably of up to 550 mm, further preferably of up to 525 mm, still further preferably of up to 500 mm. As a result, relatively stable and sufficiently compact workpiece carriers can be received or surrounded within the frame device.

Still further preferably, opposite inner edges of a frame device can have a distance from one another of 50 mm to 1000 mm, preferably of 100 mm to 900 mm, preferably of 150 mm to 850 mm, preferably of 200 mm to 800 mm, preferably of 250 mm to 750 mm, preferably of 300 mm to 700 mm, preferably of 350 mm to 650 mm, preferably of 400 mm to 600 mm, preferably of 450 mm to 550 mm, further preferably of 475 to 525 mm. Such a dimensioning allows the receiving or surrounding of workpiece carriers of suitable size for the printing within a printing device with at the same time a sufficiently stable construction and good handling. In particular, a frame device can be designed for receiving square and/or rectangular or substantially square and/or rectangular workpiece carriers.

In a still further advantageous manner, the frame device can be designed for receiving and/or enclosing a workpiece carrier in a defined and/or play-free manner. The respective position of the workpiece carrier within or on the frame device can be fixed in this way with high accuracy and can be reliably maintained for the sub-sequent process steps within the apparatus.

Additionally or alternatively, the frame device can delimit a free space for contacting the underside of a workpiece carrier received and/or enclosed therein. Good accessibility of the underside of the workpiece carrier for contacting and/or raising by a printing table is thereby ensured. In this way, direct contact between the printing table of the printing device and the respective workpiece carrier can be produced with only little effort for the respective printing operations.

In a further preferred manner, the carrying section and/or the conveying vehicle, in particular the carrying section and/or the frame device of the conveying vehicle and/or the carrying section and/or the frame device of a superstructure of the conveying vehicle, can have a workpiece carrier mounting for fixing and/or for clamping and/or for fastening a workpiece carrier in a force-fitting and/or form-fitting manner. The workpiece carrier can be fastened particularly reliably to or in the conveying vehicle by such a workpiece carrier mounting and can remain fastened within the apparatus during the transport.

The workpiece carrier mounting can particularly preferably be designed as a multi-point bearing, in particular as a 3-point bearing. Such a multi-point bearing or 3-point bearing can realize secure fastening with only little contact or small contact area with the workpiece carrier to be mounted in each case. The risk of the release of particles as a result of abrasion and therefore of the concentration in-crease of air-borne particles can be reduced further in this way. A multi-point bearing or 3-point bearing can avoid abrasion-related particle formation with high reliability.

Further preferably, the workpiece carrier mounting can be formed by at least one fixed mounting and/or by at least one locking mechanism, in particular by a plurality of fixed mountings and an oppositely arranged locking mechanism. Such a configuration is simple in terms of construction and enables simple handling or automation with a constructionally cost-effective implementation.

Further preferably, the workpiece carrier mounting can be designed for fixing and/or for clamping and/or for fastening a workpiece carrier in a force-fitting and/or form-fitting manner with a thickness of at least 0.5 mm, in particular with a thickness of at least 0.8 mm, further preferably with a thickness of at least 1 mm, even further preferably with a thickness of at least 1.5 mm, in particular of at least 2 mm or approximately 2 mm. Such a workpiece carrier mounting enables the use of workpiece carriers with a sufficiently large stability and can therefore ensure a high degree of operational reliability.

Even further preferably, the workpiece carrier mounting can be designed for fixing and/or for clamping and/or for fastening a workpiece carrier in a force-fitting and/or form-fitting manner with a thickness of up to 10 mm, in particular with a thickness of up to 8 mm, further preferably with a thickness of up to 6 mm, even further preferably with a thickness of up to 5 mm, even further preferably with a thickness of up to 4 mm, even further preferably with a thickness of up to 3 mm, even further preferably with a thickness of up to 2.5 mm, in particular of up to 2 mm. Such a workpiece carrier mounting enables the use of workpiece carriers with a particularly high degree of stability and at the same time relatively low weight.

Further preferably, the workpiece carrier mounting can be designed for fixing and/or for clamping and/or for fastening a workpiece carrier in a force-fitting and/or form-fitting manner with a thickness of 0.5 mm to 10 mm, in particular with a thickness of 0.8 mm to 8 mm, further preferably with a thickness of 1 mm to 6 mm, even further preferably with a thickness of 1 mm to 5 mm, even further preferably with a thickness of 1 mm to 4 mm, even further preferably with a thickness of 1 mm to 3 mm, even further preferably with a thickness of 1.5 mm to 2.5 mm, in particular of approximately 2 mm. A workpiece carrier mounting for workpiece carriers dimensioned in this way enables the use of workpiece carriers with a high degree of stability and at the same time relatively low weight and therefore good handling.

In a particularly preferred manner, the locking mechanism can be formed by a slide with a contact surface which is chamfered and/or inclined with respect to the sliding direction of the slide. As a result of a chamfered configuration or contact surface inclined with respect to the sliding direction of the slide, a form-fitting fastening of the respective workpiece carrier can be realized in a particularly simple and advantageous manner. In particular, a pressing force or holding-down force can be exerted on the workpiece carrier and therefore secure positioning can be ensured as a result in the closed state of the slide.

Additionally or alternatively, the locking mechanism can be formed by a slide which is under spring pretension and/or as a snap closure. A spring pretension of the slide can be directed in a closing direction of the slide, with the result that the slide is moved automatically into a closed position. Likewise, a snap closure can automatically carry out a closing movement if no manually or automatically initiated countermovement is carried out.

In a still further preferred manner, at least one fixed mounting can be formed with a contact surface which is chamfered and/or inclined with respect to the sliding direction of the slide. By means of a chamfered configuration of the fixed mounting or by means of a contact surface of the fixed mounting which is inclined with respect to the sliding direction of the slide, a form-fitting fastening of the respective workpiece carrier can be realized in a particularly simple and advantageous manner. In particular, a pressing force or holding-down force can also be exerted on the workpiece carrier and therefore secure positioning can be ensured as a result in the closed state of the slide by means of the fixed mounting.

According to a still further preferred configuration, the conveying vehicle can have at least one drive device, which is arranged on the conveying vehicle so as to travel therewith and/or which has a drive roller rolling on the transport rail and/or a drive motor for a drive roller. Such a conveying vehicle can therefore be moved or driven on the respective transport rail independently of further conveying vehicles. This allows an individual movement of the conveying vehicle within the apparatus and therefore also an individualized transport of the workpiece carrier arranged in each case on the conveying vehicle or of the screen-printed workpiece arranged in each case on the conveying vehicle.

According to a further preferred configuration, the conveying vehicle can have a superstructure for receiving a workpiece carrier for screen-printed workpieces. For this purpose, the carrying section can be arranged or formed in particular on the superstructure of the conveying vehicle. Likewise, the conveying vehicle can have a runner device having the drive device. The superstructure can preferably be arranged on the runner device of the conveying vehicle. The drive functionality and the receiving functionality for a workpiece carrier can therefore be provided by different sections or different parts of the conveying vehicle. In particular, a functional subdivision of the conveying vehicle can be realized in this way, namely into drive functionality and receiving functionality for a workpiece carrier.

In a further preferred manner, the superstructure and/or a carrying section of the superstructure and/or a frame device of the superstructure can define a receiving plane for a workpiece carrier, which extends at a distance from the runner device and/or from the drive device, in particular extends at a vertical distance.

The carrying section can be, in particular, a section of the superstructure by which a workpiece carrier can be carried or received. During the operation of the transport device, a sufficient distance between the workpiece carrier to be transported in each case and the runner device or the drive device can be maintained with such a configuration.

Insofar as particles should be released as a result of friction by the drive device and/or in the region of the runner direction, this takes place at a distance from the workpiece carrier and therefore also from the screen-printed workpieces positioned on the workpiece carrier. The risk of undesired contamination of the screen-printed workpieces can be reduced further in this way. An even further improved suitability for the use of the apparatus under clean room conditions results as a result.

In an even further preferred manner, the superstructure, in particular with a received workpiece carrier, in a plan view can cover the runner device and/or the drive device at least in sections and/or project beyond at least one side end of the runner device. As a result of such a covering and/or projecting, the risk that particles released at the level of the runner device and/or of the drive device rise to a level above the superstructure and deposit on the workpiece carrier or screen-printed workpieces located thereon can be reduced with only little constructional effort. The cleanliness conditions for the production of three-dimensional screen-printed workpieces can therefore be improved further.

It can also be advantageous if the superstructure is C-shaped and/or forms a free space for receiving a printing table of the printing device. In this case, in a cross-section of the superstructure the surface center can be arranged laterally offset with respect to the center of gravity of the superstructure. Such a configuration is simple to implement in terms of construction and at the same time ensures that a workpiece carrier arranged on the superstructure can be contacted with a high degree of safety and stability by a printing table, in particular for carrying out a printing operation.

In a still further preferred manner, the superstructure can have a base section for fastening to the runner device, a carrying section for receiving a workpiece carrier and a connecting section for connecting the carrying section to the base section, wherein a free space for receiving a printing table and/or for contacting the underside of a workpiece carrier can preferably be formed between the base section and the carrying section and/or can be delimited laterally by the connecting section. On the one hand, a stable construction of the superstructure is thereby ensured, with good accessibility of a workpiece carrier positioned on the superstructure from the underside thereof at the same time.

In a further preferred manner, the height of a free space and/or a distance between the base section and the carrying section can be up to 150 mm, further preferably up to 140 mm, further preferably up to 130 mm, further preferably up to 120 mm, further preferably up to 110 mm, further preferably up to 100 mm, further preferably up to 95 mm, in particular up to 90 mm or approximately 90 mm. By means of such a configuration, a sufficiently compact construction can be ensured and at the same time a temporary reception of apparatus components in the free space between the base section and the carrying section can advantageously be ensured.

In a further preferred manner, the height of a free space and/or a distance between the base section and the carrying section can be at least 20 mm, further preferably at least 30 mm, further preferably at least 40 mm, further preferably at least 50 mm, further preferably at least 60 mm, further preferably at least 70 mm, further preferably at least 80 mm, further preferably at least 85 mm, in particular at least 90 mm. As a result, a free space between the base section and the carrying section can advantageously serve for temporarily receiving apparatus components, with reduced risk of collisions.

In a further preferred manner, the height of a free space and/or a distance between the base section and the carrying section can be between 20 mm and 150 mm, further preferably between 30 mm and 140 mm, further preferably between 40 mm and 130 mm, further preferably between 50 mm and 120 mm, further preferably between 60 mm and 110 mm, further preferably between 70 mm and 100 mm, further preferably between 80 mm and 100 mm, further preferably between 85 mm and 95 mm, in particular approximately 90 mm. Such a dimensioning of the free space or of the distance between the base section and the carrying section ensures a compact and robust construction with at the same time a low risk of collision in the course of the arrangement of apparatus components within the free space.

It can also be advantageous if the superstructure and/or the carrying section of the superstructure is mounted so as to be movable relative to the runner device in the vertical direction and/or is arranged so as to be adjustable in height. Additionally or alternatively, the superstructure and/or the carrying section of the superstructure can be arranged so as to be raisable relative to the runner device by a printing table of the printing device.

By means of such a configuration, the superstructure and/or the carrying section of the superstructure can be moved in the vertical direction or raised relative to the runner device for carrying out a printing operation. Consequently, when carrying out a printing operation, the superstructure and/or the carrying section of the superstructure can be raised together with the respective workpiece carrier relative to the runner device and can thereby be positioned with increased stability. Forces occurring when carrying out a printing operation can be absorbed in a suitable manner by a printing table in a raised state, whereas a transmission of force or absorption of force by the runner device and/or by the transport rail can be avoided.

A vertically movable arrangement or mounting of the superstructure relative to the runner device can be provided in particular independently of a vertical adjustability of the superstructure and/or of the carrying section of the superstructure relative to the runner device.

A vertical adjustability of the superstructure and/or of the carrying section of the superstructure relative to the runner device can be ensured in particular by at least one adjusting screw, in particular by a plurality of adjusting screws. As a result, a suitable orientation and adjustment of the height of the superstructure and/or of the carrying section can be ensured. A lifting operation of the superstructure and/or of the carrying section of the superstructure by a printing table can therefore be carried out starting from a correctly set initial height of the superstructure and/or of the carrying section of the superstructure.

In a preferred manner, a vertical adjustability of at least 5 mm, preferably of at least 10 mm, further preferably of at least 15 mm, still further preferably of at least 20 mm, still further preferably of at least 30 mm can be provided. As a result, a sufficiently precise adjustment of the height of the superstructure and/or of the carrying section of the superstructure relative to the runner device can be ensured even in the case of relatively large adjusting requirements.

Furthermore, a vertical adjustability of at most 30 mm, preferably of at most 20 mm, further preferably of at most 15 mm, still further preferably of at most 10 mm can be provided. Such a configuration can be brought about with only a small installation space and at the same time ensures a high degree of stability.

According to a further preferred configuration, a stroke and/or a vertical movability of the superstructure and/or of the carrying section of the superstructure relative to the runner device can be delimited. Such a limitation can be formed in particular by a mechanical stroke-limiting device, particularly preferably by a stroke-limiting claw. In this way, it can be ensured that the superstructure or the carrying section of the superstructure is not undesirably detached from the runner device. A high degree of operational reliability is ensured as a result.

In a further preferred manner, a stroke-limiting device can delimit the stroke of the superstructure and/or of the carrying section of the superstructure relative to the runner device at a stroke distance of at most 20 mm, further preferably of at most 15 mm, still further preferably of at most 10 mm, still further preferably of at most 5 mm.

In a further preferred manner, the stroke-limiting device and/or the stroke-limiting claw can be arranged on the superstructure and/or engage behind the runner device in a form-fitting manner. By raising the superstructure, the stroke-limiting device and/or the stroke-limiting claw can be brought into form-fitting contact engagement with the runner device. In this way, a suitable transport securing means for the superstructure can be provided with only little constructional effort.

According to a further preferred configuration, the superstructure can be fastened to the runner device and/or guided in a vertically movable manner via a multi-point bearing, in particular via a 3-point bearing or via a 4-point bearing. At least one bearing between the superstructure and the runner device can be formed by a bearing mandrel and a mandrel receptacle for the bearing mandrel. For the transport, a sufficiently secure positioning of the superstructure on the runner device can be ensured in this way. In particular, the superstructure can be, in a set-down position or in a set-down position on the runner device, positioned in a play-free manner relative to the runner device in a horizontal direction. At the same time, a secure raising of the superstructure can be ensured by a vertically movable guide.

In a still further preferred configuration, the multi-point bearing and/or at least one bearing between the superstructure and the runner device can be designed for self-centering and/or self-alignment of the superstructure relative to the runner device. During the setting-down or lowering of the superstructure on the runner device, the correct positioning can be ensured as a result with a high degree of reliability and little effort.

In a further advantageous manner, at least one bearing mandrel can taper conically and/or at least one mandrel receptacle can taper conically. A self-centering of the superstructure relative to the runner device can be brought about as a result in a constructionally simple manner. In a still further advantageous manner, it is possible for at least one mandrel receptacle to taper conically in a complementary manner to the bearing mandrel, as a result of which a planar contacting between the respectively tapering and mutually facing surfaces is realized. Likewise, at least one mandrel receptacle can have a conicity deviating from the respective bearing mandrel, as a result of which a particularly small contact surface between the mandrel receptacle and the respective bearing mandrel can result.

Further preferably, a mandrel receptacle can be formed on the underside of an adjusting screw for the vertical adjustment of the superstructure relative to the runner device. Such an adjusting screw can be screwed in particular into a base section of the superstructure and arranged in a screwable manner for the vertical adjustment.

According to a still further preferred configuration, the conveying vehicle can have at least one supporting device for supporting on the transport rail, in particular a supporting roller rolling on the transport rail for supporting the conveying vehicle. Additionally or alternatively, the conveying vehicle can be supported on the transport rail via roller contact points, in particular exclusively via roller contact points of a drive roller and/or of a supporting roller or via roller contact points of a plurality of drive rollers and/or supporting rollers. In this way, a secure and stable guidance of the conveying vehicle on the transport rail and therefore a particularly secure transport of workpiece carriers and/or screen-printed workpieces can be brought about.

The transport rail can preferably be designed as a monorail and/or have a plurality of monorail sections connected to one another, in particular monorail sections connected to one another in the longitudinal direction. The transport rail can still further preferably be assembled in a modular manner and/or consist of a plurality of rail modules.

A monorail is provided in particular as a so-called single rail or for forming an individual travel path for the respective conveying vehicle. In the case of a configuration as a monorail, a conveying vehicle is supported only on a transport rail forming an individual travel path. A further transport rail running in parallel for simultaneously supporting the respective conveying vehicle is therefore not provided in the case of a monorail. Such a monorail ensures a compact and stable construction and numerous transport configurations and route profiles can be realized by connecting a plurality of monorail sections and/or a plurality of rail modules with only little effort and at low costs.

Still further preferably, the conveying vehicle can be arranged on the transport rail so as to engage around it on multiple sides, in particular so as to engage around it on three sides, along a longitudinal section of the transport rail. The stability of the conveying vehicle on the transport rail can be advantageously improved as a result.

In a still further preferred manner, the conveying vehicle can have at least one position sensor and/or one distance sensor. A reliable position monitoring and/or position control of the conveying vehicle can take place as a result. In particular, a collision of two conveying vehicles can be avoided by such a configuration.

Furthermore, the conveying vehicle can be designed for communication with a control module arranged on the transport rail. The communication via such a control module makes it possible to transmit signals to the conveying vehicle in order to define the desired transport path and/or the transport speed or the transport course in each case.

Consequently, according to a further preferred configuration, at least one control module can be arranged on the transport rail. Such a control module can be arranged for communication with a superordinate apparatus controller and/or for communication with at least one conveying vehicle for controlling the transport path and/or the transport speed of the respective conveying vehicle. The respective conveying vehicle, in particular a plurality of conveying vehicles, can be, via such a control module, controlled in a particularly advantageous manner, namely by a superordinate apparatus controller. Transport paths and/or transport speeds of a plurality of conveying vehicles can be coordinated with one another. Likewise, transport paths and/or transport speeds of conveying vehicles can be adapted to the processing and process times for the respective screen-printed workpieces.

Additionally or alternatively, at least one control unit, in particular control cam, for controlling the speed of a conveying vehicle can be arranged on the transport rail. Such a control unit can preferably be designed exclusively for controlling the speed of a conveying vehicle. In this way, the transport speed and/or the transport course of a conveying vehicle can be influenced with only little equipment effort.

According to a further preferred configuration, at least one position sensor and/or one distance sensor can be arranged on the transport rail. Collisions of conveying vehicles, in particular of adjacent conveying vehicles or conveying vehicles arranged one behind the other on the transport rail, can be avoided with high reliability by position and/or distance sensors.

Still further preferably, the transport device can have a collision monitoring system for avoiding collisions between conveying vehicles. In this case, the collision monitoring system can be set up for signal processing of distance and/or position sensors and/or of control modules and/or control units of the transport rail. The operational reliability of the apparatus can be further improved in this way and the degree of automation can be further increased.

According to a still further preferred configuration, the superstructure, in particular the carrying section and/or the frame device, can have support sections or at least one support section for contacting and/or raising the superstructure by a printing table of the printing device. Such support sections can preferably be formed on an underside of the carrying section and/or of the frame device. By means of such support sections, the superstructure of the conveying vehicle, in particular the carrying section and/or the frame device, can be contacted and raised by a printing table independently of the respective workpiece carrier. The formation on the underside of the carrying section allows good accessibility for a printing table and therefore an overall robust and cost-effective construction.

In a still further preferred manner, the support sections can be formed and/or arranged for contacting by a printing table in a manner load-free for the workpiece carrier mounting. The superstructure can be contactable and/or liftable via the support sections by a printing table in a manner load-free for the workpiece carrier mounting. The contacting and raising of the superstructure and at the same time also of the workpiece carrier by a printing table of a printing device can therefore take place in a manner load-free or free of additional force effects on a workpiece carrier mounting of the superstructure. The secure fixing of the respective workpiece carrier in or on the superstructure of the conveying vehicle is not impaired by contacting, raising and lowering of the superstructure together with the workpiece carrier.

According to a still further preferred configuration, the printing device can have a liftable printing table and/or a printing table designed as a vacuum printing table, in particular a liftable vacuum printing table.

A printing table designed as a vacuum printing table can have in particular grooves and/or openings for generating a vacuum and/or a negative pressure on the underside of a workpiece carrier to be fixed and located on the printing table. With a vacuum printing table, a workpiece carrier can be fixed with little effort and with a high degree of safety, in particular temporarily and for carrying out a printing operation.

According to a still further preferred configuration, the printing table of the printing device can be designed for planar contacting of a workpiece carrier received in a conveying vehicle. Additionally or alternatively, the printing table can be designed for contacting support sections of the superstructure of the conveying vehicle. A particularly secure and reliable raising of the superstructure and also of the workpiece carrier positioned in each case on the superstructure can be brought about in this way.

In a particularly preferred manner, the printing table of the printing device can be designed to raise the superstructure via the support sections in a manner load-free for the workpiece carrier mounting, in particular with simultaneous contacting of the workpiece carrier. An undesired release of the workpiece carrier from the workpiece carrier mounting can be avoided as a result and therefore a high degree of operational reliability can be ensured. A raising in a manner load-free for the workpiece carrier mounting can additionally prevent undesired relative movements between the workpiece carrier mounting and the workpiece carrier held in each case therein, with the result that undesired abrasion and therefore the release of air-floating particles can also be prevented.

In a still further preferred manner, in a plan view of the transport device, the center of gravity of the conveying vehicle can be arranged offset with respect to the transport rail. In particular, the center of gravity of the conveying vehicle can be arranged offset with respect to the transport rail in the horizontal direction. In this way, for predefined cornering of the conveying vehicle, in particular in a predefined cornering direction, the risk of tilting movements of the conveying vehicle can be advantageously reduced. This applies particularly in the case of a mounting of the superstructure and/or of the carrying section of the superstructure movable in the vertical direction relative to the runner device. A vertically movable mounting of the superstructure and/or of the carrying section of the superstructure can lead to tilting movements in cornering, which tilting movements can be advantageously counteracted by an offset arrangement of the center of gravity of the conveying vehicle, in particular an arrangement offset in the horizontal direction relative to the transport rail. An undesired release or falling down of the workpiece carrier carried in each case can be prevented overall as a result.

In a still further preferred manner, in a plan view of the transport device, the center of gravity of the conveying vehicle can be arranged offset with respect to the transport rail by at least 10 mm, preferably by at least 15 mm, further preferably by at least 20 mm, still further preferably by at least 25 mm, still further preferably by at least 30 mm, still further preferably by at least 40 mm, still further preferably by at least 50 mm, still further preferably by at least 60 mm, still further preferably by at least 70 mm or at least 80 mm. Tilting movements in cornering can be particularly reliably avoided as a result.

In a still further preferred manner, in a plan view of the transport device, the center of gravity of the conveying vehicle can be arranged offset with respect to the transport rail by up to 80 mm, preferably by up to 70 mm, further preferably by up to 60 mm, still further preferably by up to 50 mm, still further preferably by up to 40 mm, still further preferably by up to 30 mm, still further preferably by up to 25 mm, still further preferably by up to 20 mm, still further preferably by up to 15 mm, in particular by up to 10 mm. As a result of such an arrangement of the center of gravity, a sufficient stability on the transport rail can be ensured even for straight-ahead travel and for a stationary position of the conveying vehicle.

Further preferably, in a plan view of the conveying vehicle, the center of gravity of the superstructure can be arranged offset with respect to the center of gravity of the runner device. In particular, the center of gravity of the superstructure can be arranged offset with respect to the center of gravity of the runner device in the horizontal direction. Such a configuration can reduce the risk of tilting movements of the conveying vehicle still further. As a result of the offset center of gravity of the superstructure, a moment can be produced in a particularly suitable manner, which moment can counteract any tilting movements of the superstructure in cornering with a high degree of safety. This applies again in a particular manner in the case of a mounting of the superstructure and/or of the carrying section of the superstructure movable in the vertical direction relative to the runner device. A vertically movable mounting of the superstructure and/or of the carrying section of the superstructure can lead to tilting movements in cornering, which tilting movements can be advantageously counteracted by an offset arrangement of the center of gravity of the superstructure, in particular an arrangement offset with respect to the center of gravity of the runner device in the horizontal direction.

In a still further preferred manner, in a plan view of the conveying vehicle, the center of gravity of the superstructure can be arranged offset with respect to the center of gravity of the runner device by at least 10 mm, preferably by at least 15 mm, further preferably by at least 20 mm, still further preferably by at least 25 mm, still further preferably by at least 30 mm, still further preferably by at least 40 mm, still further preferably by at least 50 mm, still further preferably by at least 60 mm, still further preferably by at least 70 mm or at least 80 mm. Tilting movements in cornering can again be particularly reliably avoided as a result.

In a still further preferred manner, in a plan view of the conveying vehicle, the center of gravity of the superstructure can be arranged offset with respect to the center of gravity of the runner device by up to 80 mm, preferably by up to 70 mm, further preferably by up to 60 mm, still further preferably by up to 50 mm, still further preferably by up to 40 mm, still further preferably by up to 30 mm, still further preferably by up to 25 mm, still further preferably by up to 20 mm, still further preferably by up to 15 mm, in particular by up to 10 mm. As a result of such an arrangement of the center of gravity, a sufficient stability on the transport rail can again be ensured for straight-ahead travel and for a stationary position of the conveying vehicle.

In a still further preferred manner, the conveying vehicle and/or the superstructure of the conveying vehicle and/or the frame device of the conveying vehicle can define and/or have a receiving center point for a workpiece carrier. Likewise, such a receiving center point for a workpiece carrier can be defined by a workpiece carrier mounting. In the case of a workpiece carrier arranged and/or fixed on the conveying vehicle and/or on the superstructure and/or in the frame device of the superstructure, the center point of the workpiece carrier can be located in particular on the receiving center point and/or in a plan view of the conveying vehicle in alignment with the receiving center point.

In a further preferred manner, in a plan view of the conveying vehicle, the receiving center point for a workpiece carrier can be arranged offset with respect to the transport rail. In particular, in the case of the conveying vehicle, the receiving center point for a workpiece carrier can be arranged offset with respect to the transport rail in the horizontal direction. In this way, particularly good accessibility of the workpiece carrier or contactability of the workpiece carrier by a printing table can be ensured.

In a still further preferred manner, in a plan view of the conveying vehicle, the receiving center point for a workpiece carrier can be arranged offset with respect to the runner device. In particular, in the case of the conveying vehicle, the receiving center point for a workpiece carrier can be arranged offset with respect to the runner device in the horizontal direction. In this way, particularly good accessibility of the workpiece carrier or contactability of the workpiece carrier by a printing table can also be ensured.

According to a further preferred configuration, the transport device and/or the rail system can be formed as a transport circuit, and/or can be set up for the automated transport of at least one screen-printed workpiece and/or of a workpiece carrier in the circuit between the printing device and a position spaced apart from the printing device. Such a design ensures a high degree of productivity. Different devices or stations within the entire apparatus can advantageously be connected to one another by such a transport device. A substantially interruption-free production sequence with successive steps in the respective devices or stations can be ensured as a result.

In a further preferred manner, a plurality of printing devices, between which the transport device runs and/or between which a screen-printed workpiece and/or a workpiece carrier can be transported by means of the transport device, can be provided. The productivity or also the production flexibility can be further increased by a plurality of or different printing devices.

In particular, a plurality of printing devices can be provided to print different materials or material compositions successively and with only little effort and/or to use different printing screens or printing stencils during the production of an individual screen-printed workpiece. For the production of differently designed printing layers of an individual screen-printed workpiece, in such an arrangement, a complicated conversion or adaptation of the respective printing devices can be avoided.

In a still further preferred manner, at least one drying device can be provided for at least one screen-printed workpiece, in particular a plurality of drying devices. After the application of a printing layer, a reliable drying of the printing layer executed last can be carried out by such a drying device, in order then again to apply the next printing layer to the screen-printed workpiece.

The at least one drying device can preferably be formed for the continuous drying pass-through and/or for the stationary drying. The drying device can be formed in particular with a drying section for the continuous drying pass-through of screen-printed workpieces and/or workpiece carriers.

A continuous drying pass-through permits an overall continuous production process for a plurality of screen-printed workpieces or with a plurality of workpiece carriers on which screen-printed workpieces can be arranged. Both the printing and the drying can be carried out substantially without interruption by a continuous drying pass-through, as a result of which the overall productivity of the apparatus can be further increased.

A drying device for the stationary drying can be constructed in a particularly compact and space-saving manner. In a stationary drying, a screen-printed workpiece or a workpiece carrier with a screen-printed workpiece arranged thereon can be temporarily positioned or stopped in order to carry out a drying step.

According to a still further preferred configuration, the transport device can be designed for the transport of a screen-printed workpiece and/or of a workpiece carrier towards and/or away from the drying device and/or through the drying device. Additionally or alternatively, the transport device can be designed for the transport of a screen-printed workpiece and/or of a workpiece carrier between the printing device and the drying device. In this way, workpiece carriers with screen-printed workpieces can be supplied with only little effort for drying and subsequently moved away again, for example for carrying out further printing steps.

Furthermore preferably, the transport device can run through the drying device, with the result that manual handling of the workpiece carriers can be completely avoided. Additionally or alternatively, the transport device can be formed separately and/or independently of the drying device. As a result, an overall modular construction can be made possible or further promoted. The transport device can be configured independently of the drying device and can be arranged for connecting the transport device to at least one further device or position within the apparatus.

Despite the transport device being formed separately and/or independently of the drying device, it can be adapted to the drying device, in particular in order to enable a reliable transport of a workpiece carrier and/or of a screen-printed workpiece towards and/or away from the printing device.

In a further preferred manner, the workpiece carrier can be movable by means of the transport device independently of the printing device or of the printing table plate through the drying device, in particular can be movable in an automated manner through the drying device.

In a particularly preferred manner, the drying device can be designed for the drying of at least one screen-printed workpiece and for the simultaneous cooling of a workpiece carrier carrying the respective screen-printed workpiece to be dried. In this way, on the one hand a suitable drying of the respective screen-printed workpiece or of the last printed layer of the screen-printed workpiece can be carried out. At the same time, the workpiece carrier can be kept at a relatively low temperature by the cooling. An undesired heating of the workpiece carrier and therefore also an excessively strong heating of already printed and previously dried layers of the respective screen-printed workpiece can be avoided as a result. The process reliability can be further improved in this way.

In a particularly preferred manner, the drying device can have a drying section for the drying of screen-printed workpieces and a cooling section for the cooling of workpiece carriers. The drying device can preferably be divided into a drying section and a cooling section. Such a subdivision can avoid a mutual impairment of the drying and cooling functionality or reduce it to a low degree.

In a preferred manner, a subdivision of the drying device into a drying section and a cooling section can be defined by a plane on which a workpiece carrier is arranged during positioning in the drying device and/or during transport through the drying device.

Furthermore, at least one nozzle device and/or perforated plate for the supply of a cooling air flow, in particular of compressed cooling air, can preferably be arranged within the cooling section. In particular, a drying section can be arranged above a nozzle device and/or perforated plate for the supply of a cooling air flow. As a result, effective cooling is made possible with only little constructional effort.

According to a still further preferred configuration, the drying device can be designed for convection drying of a screen-printed workpiece and/or for blowing air cooling of workpiece carriers. In particular, the drying device can be designed for convection drying of a screen-printed workpiece with simultaneous blowing air cooling of the workpiece carrier carrying the respective screen-printed workpiece.

Convection drying and also simultaneous blowing air cooling can be realized relatively simple and with little constructional effort. In this case, in particular a simultaneousity of the convection drying of a screen-printed workpiece and blowing air cooling of the respective workpiece carrier can ensure improved operational reliability since, despite the convection drying, an undesirably strong heating of the workpiece carrier and therefore also a heating of the layers already printed on the workpiece carrier can be reliably avoided.

Additionally or alternatively, the drying device can have contact cooling for a workpiece carrier, preferably contact cooling for a workpiece carrier that travels therewith. Such contact cooling ensures a very low impairment of the drying functionality.

In a still further preferred manner, the drying device can be set up to activate and deactivate cooling positions depending on the position of a workpiece carrier within the drying device, in particular to activate cooling positions below the respective current position of a workpiece carrier. In this way, cooling can take place exclusively in the region of the current position of a workpiece carrier. The energy consumption for the cooling and also for the drying can be kept low overall as a result since unfavorable impairments between the drying and the cooling can also be avoided.

Further preferably, a cooling position can be set up to be activated by selective actuation of individual or a plurality of cooling nozzles and/or compressed air openings in and/or on a nozzle device and/or perforated plate.

Accordingly, a cooling position can be set up to be deactivated by selective actuation of individual or a plurality of cooling nozzles and/or compressed air openings in and/or on a nozzle device and/or perforated plate. In particular, a deactivation can be carried out by selective shutting down or blocking of one or a plurality of previously selectively actuated cooling nozzles and/or compressed air openings in and/or on a nozzle device and/or perforated plate.

According to a further preferred configuration, the drying device can have at least one air filter and/or a plurality of nozzles for filtered drying air. The risk of air contamination and therefore also of undesired particle introduction into the respective screen-printed workpieces can therefore be reduced further.

Additionally or alternatively, the drying device can be formed as a circulating air dryer and/or for continuous and/or periodic supply of fresh air and/or room air.

Such a circulating air dryer can be operated with relatively low energy effort and at the same time ensure effective drying of screen-printed workpieces.

In a still further preferred configuration, the drying device can be designed to direct a drying air flow onto a screen-printed workpiece, in particular at an angle to a transport axis formed by the transport rail or at an angle to the transport direction of the respective screen-printed workpiece and/or of the respective workpiece carrier and/or transversely to the transport direction thereof and/or on the upper side onto the respective screen-printed workpiece.

A transport axis or transport direction of the respective screen-printed workpiece and/or of the respective workpiece carrier runs in particular along the longitudinal extent of the transport rail. If a drying air flow is directed at an angle onto a screen-printed workpiece, this can be a direction of the drying air flow which differs from the transport axis or transport direction of the respective screen-printed workpiece and/or of the respective workpiece carrier. It can therefore be a direction of the drying air flow which is inclined with respect to the transport axis or the transport direction of the respective screen-printed workpiece and/or of the respective workpiece carrier, namely is inclined by a specific or predefined angle. A direction of the drying air flow transversely to the transport axis of the transport rail or transversely to the transport direction of the respective screen-printed workpiece and/or of the respective workpiece carrier runs in particular at a right angle or at an angle of 90° relative to the transport axis or relative to the transport direction of the respective screen-printed workpiece and/or of the respective workpiece carrier.

By such a configuration of the drying device, on the one hand a suitable drying by a drying air flow can be ensured, with at the same time a low risk of undesired relative movements between the screen-printed workpiece and the respective workpiece carrier. In addition, by means of such a drying air flow, a subdivision into cooling section and drying section can advantageously be defined by a plane on which a workpiece carrier is arranged during positioning in the drying device and/or during transport through the drying device.

Further preferably, the drying device can be formed to extract air flows in such a way that an extraction air flow is established at an angle to a transport axis formed by the transport rail or at an angle to the transport direction of the respective screen-printed workpiece and/or of the respective workpiece carrier, in particular transversely to the transport direction and/or in a horizontal direction from the respective screen-printed workpiece and/or workpiece carrier.

If air flows are extracted in such a way that, on account of the extraction, an extraction air flow is established at an angle to a transport axis formed by the transport rail or at an angle to the transport direction of the respective screen-printed workpiece and/or of the respective workpiece carrier, this can be a direction which differs from the transport axis or transport direction of the respective screen-printed workpiece and/or of the respective workpiece carrier. It can therefore be a direction which is inclined with respect to the transport axis formed by the transport rail or with respect to the transport direction of the respective screen-printed workpiece and/or of the respective workpiece carrier, namely is inclined by a specific or predefined angle. A direction of the extraction air flow transversely to the transport axis or transport direction of the respective screen-printed workpiece and/or of the respective workpiece carrier runs in particular at a right angle or at an angle of 90° relative to the transport axis or transport direction of the respective screen-printed workpiece and/or of the respective workpiece carrier.

By means of such a configuration, an undesired impairment between a drying air flow directed onto the workpiece carrier and a drying air flow extracted or directed away from the workpiece carrier can be avoided.

Furthermore, the drying device can advantageously be set up to direct extracted drying air and/or cooling air again as drying air onto a screen-printed workpiece to be dried. In this case, extracted drying air and/or cooling air can be directed completely or at least partially again as drying air onto a screen-printed workpiece to be dried. In the latter case, the drying device can be designed and/or configured for a so-called mixed operation. Extracted drying air and/or cooling air can be mixed with fresh air or newly supplied air before the respective mixed air quantities are directed onto a screen-printed workpiece to be dried. Energy-efficient operation can be ensured overall in this way.

Furthermore, the drying device can advantageously have a heat exchanger, in particular for heat recovery of extracted drying air and/or cooling air. Consequently, extracted drying air and/or cooling air can be supplied to a heat exchanger in order to use recovered heat energy for heating a drying air flow. When such a heat exchanger is used, extracted drying air and/or cooling air, after it has been guided past the heat exchanger, can be directed at least partially or also completely into the open or out of the drying device.

Such a configuration can on the one hand ensure energy-efficient operation. At the same time, however, the risk of particle contamination is reduced, since extracted drying air and/or cooling air which are possibly already contaminated with particles on account of being guided past the respective screen-printed workpieces and/or the workpiece carriers can be directed out of the drying device. In such a configuration, energy recovery requires in particular no circulation of the drying air and/or cooling air per se, but at most the heat energy of the drying air and/or cooling air can be recovered.

According to a still further preferred configuration, when a conveying vehicle is positioned within the drying device, the nozzle device and/or perforated plate can project at least in sections into a free space of the superstructure for receiving a printing table and/or between the base section and the carrying section of the superstructure and/or at least in sections below a workpiece carrier received by the conveying vehicle. In this way, a cooling air flow can be directed onto an underside of the workpiece carrier with particularly little effort. At the same time, as a result, the runner device can be covered at least in sections by the nozzle device and/or perforated plate.

The risk of contamination of the screen-printed workpieces by particles which are released in the region or at the level of the runner device or are present in a freely floating manner there can be reduced by such a configuration.

Still further preferably, the drying device can have an opening on at least one longitudinal end, which opening is formed at least in sections in a complementary manner, in particular in a geometrically complementary manner, to the cross-sectional shape and/or in a complementary manner, in particular in a geometrically complementary manner, to a cross-sectional sectional shape of the conveying vehicle. The respective opening on the longitudinal end can therefore be relatively small. An air exit from the opening can therefore be limited to a low degree and energy-efficient operation of the drying device can therefore be ensured.

According to a still further preferred configuration, the apparatus can have a sluice device for the sluice in and/or sluice out of conveying vehicles. The replacement, the supplementation and/or the removal of conveying vehicles, for example for the purpose of maintenance and/or repair work and/or for the purpose of reconfiguring the transport device, can be carried out as a result with little handling effort and high operational reliability.

According to a still further preferred configuration, the apparatus can have at least one detection module for the detection of sluiced-in and/or to be sluiced-out conveying vehicles of the transport device. The monitoring of the stock of conveying vehicles within the apparatus can be ensured as a result with high reliability and the supplementation and/or the removal of conveying vehicles can take place as a function of the detection of sluiced-in or sluiced-out conveying vehicles.

According to a still further preferred configuration, the apparatus can have at least one positioning and/or removal apparatus for the positioning of workpiece carriers on a conveying vehicle and/or for the removal of a workpiece carrier from a conveying vehicle. Manual handling of workpiece carriers can therefore be completely avoided or at least reduced to a low degree. The process reliability is therefore further improved.

A further independent aspect of the present invention relates to an apparatus for producing three-dimensional screen-printed workpieces, in particular a 3D screen printing machine, having a printing device for the layerwise production of at least one screen-printed workpiece in a plurality of printing operations and having a drying device which is designed for drying at least one screen-printed workpiece and for simultaneously cooling a workpiece carrier carrying the respective screen-printed workpiece to be dried.

A still further independent aspect of the present invention relates to a drying device for three-dimensional screen-printed workpieces, in particular for an apparatus according to one of the preceding aspects, having a drying section for the drying of at least one screen-printed workpiece and having a cooling section for the simultaneous cooling of a workpiece carrier carrying the respective screen-printed workpiece to be dried.

A still further independent aspect of the present invention relates to an apparatus for producing three-dimensional screen-printed workpieces, in particular a 3D screen-printing machine, having a printing device for the layerwise production of at least one screen-printed workpiece in a plurality of printing operations and having a transport device for the automated transport of at least one screen-printed workpiece and/or of a workpiece carrier towards and/or away from the printing device, wherein the transport device has at least one transport rail and a conveying vehicle, arranged movably on the transport rail, for at least one screen-printed workpiece and/or at least one workpiece carrier.

A still further independent aspect of the present invention relates to an apparatus for producing three-dimensional screen-printed workpieces, in particular according to one of the preceding aspects and/or a 3D screen-printing machine, having a printing device for the layerwise production of at least one screen-printed workpiece in a plurality of printing operations and having a transport device for the automated transport of at least one screen-printed workpiece and/or of a workpiece carrier towards and/or away from the printing device, wherein the transport device has a workpiece carrier mounting for the fixing and/or for the clamping of a workpiece carrier.

A still further independent aspect of the present invention relates to a transport device, in particular for the transport in an apparatus for producing three-dimensional screen-printed workpieces, having a conveying vehicle for the automated transport of at least one screen-printed workpiece and/or of a workpiece carrier, wherein the conveying vehicle has a carrying section for the at least form-fitting and/or force-fitting and/or material-bonded reception of a workpiece carrier and/or a workpiece carrier mounting for the fixing and/or for the clamping of a workpiece carrier.

A still further independent aspect of the present invention relates to a method for producing three-dimensional screen-printed workpieces, in particular using an apparatus according to one of the preceding aspects, in which at least one screen-printed workpiece is produced layerwise in a plurality of printing operations by a printing device and in which at least one screen-printed workpiece and/or a workpiece carrier with a screen-printed workpiece is transported in an automated manner towards and/or away from the printing device by a transport device, wherein the transport by the transport device is carried out by means of a conveying vehicle on a transport rail.

By the transport by means of a conveying vehicle on a transport rail, relative movements of surfaces arranged in a contacting manner can be avoided or reduced to a low degree. Particle abrasion by the travel of the conveying vehicle on the transport rail can therefore be avoided or significantly reduced with such a method. Improved cleanliness conditions can therefore be created for the individual production steps. Undesired contamination of the screen-printed workpieces to be produced in each case on account of particles arising as a result of friction can be prevented with a method according to the invention. Consequently, screen-printed workpieces with high cleanliness or hygiene requirements can be produced with a method according to the invention.

A still further independent aspect of the present invention relates to a method for producing three-dimensional screen-printed workpieces, in particular having an apparatus according to one of the preceding aspects, in which at least one screen-printed workpiece is produced layerwise in a plurality of printing operations by a printing device, in which the screen-printed workpiece is dried by a drying device and a workpiece carrier carrying the screen-printed workpiece is simultaneously cooled.

A still further independent aspect of the present invention relates to a method for producing three-dimensional screen-printed workpieces, in particular using an apparatus according to one of the preceding aspects, in which at least one screen-printed workpiece is produced layerwise in a plurality of printing operations by a printing device and in which at least one screen-printed workpiece on a workpiece carrier is transported in an automated manner towards and/or away from the printing device by a transport device, wherein the workpiece carrier is received at least in a form-fitting manner on and/or in a carrying section for the transport by the transport device.

A still further independent aspect of the present invention relates to a method for producing three-dimensional screen-printed workpieces, in particular using an apparatus according to one of the preceding aspects, in which at least one screen-printed workpiece is produced layerwise in a plurality of printing operations by a printing device and in which at least one screen-printed workpiece on a workpiece carrier is transported in an automated manner towards and/or away from the printing device by a transport device, wherein the workpiece carrier is fixed and/or clamped in a workpiece carrier mounting for the transport by the transport device. The details and independent aspects described above with regard to the apparatus, including the independent aspects relating to a drying device and relating to a transport device, apply in the same manner to the methods according to the invention described above according to the further independent aspects. Likewise, the details and independent aspects described above with regard to the apparatus also apply in the same manner to the independent aspects described above relating to a drying device and also relating to a transport device.

The invention is described below by way of example on the basis of advantageous embodiments with reference to the appended figures.

It is shown in each case schematically:

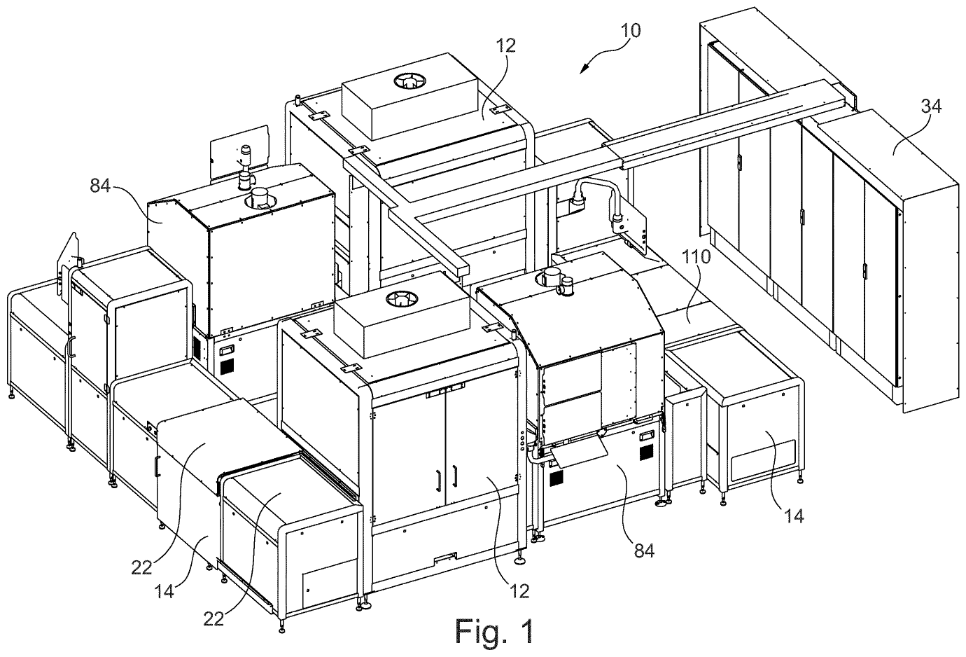

FIG. 1 a perspective view of an apparatus according to the invention according to an embodiment,

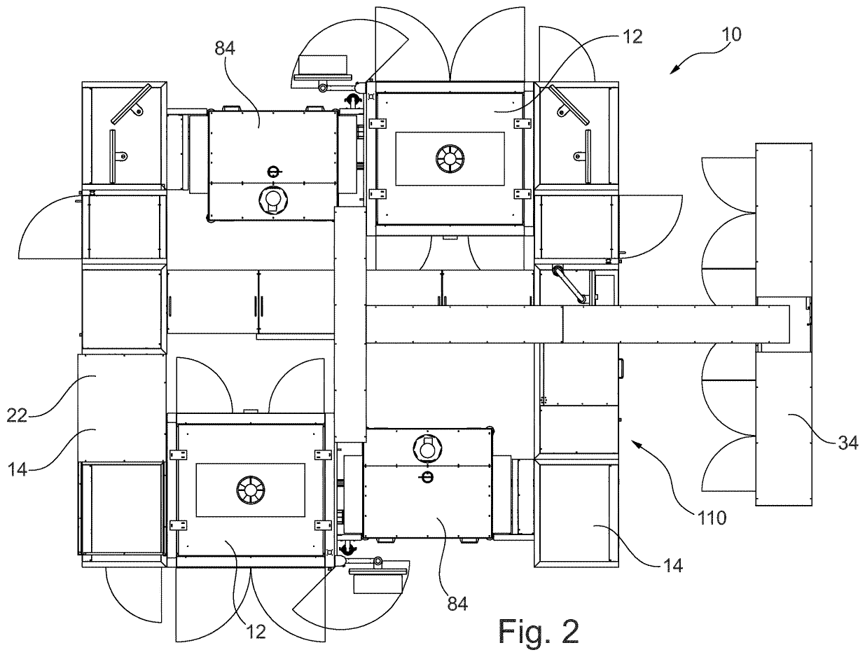

FIG. 2 a plan view of the apparatus of FIG. 1,

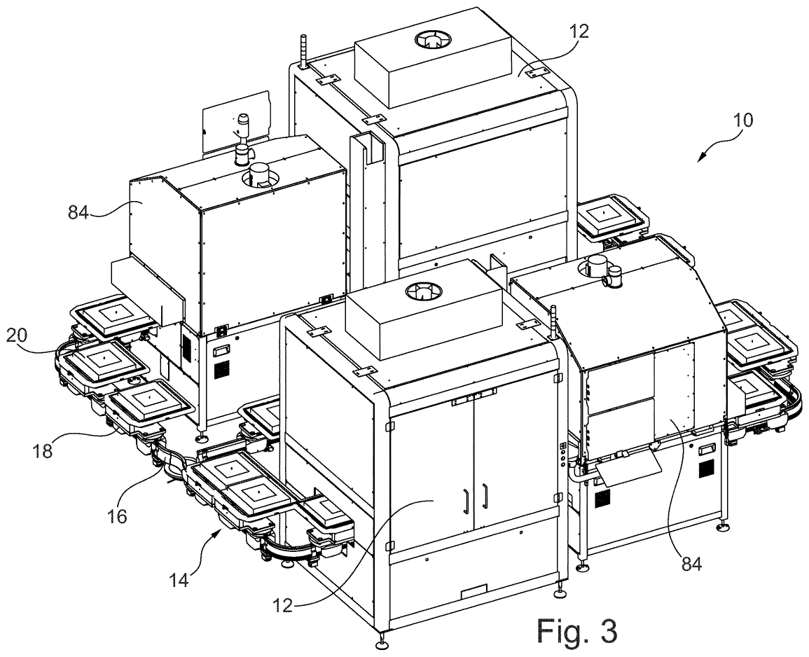

FIG. 3 a perspective view of the apparatus of FIG. 1 with open representation of the transport device without housing,

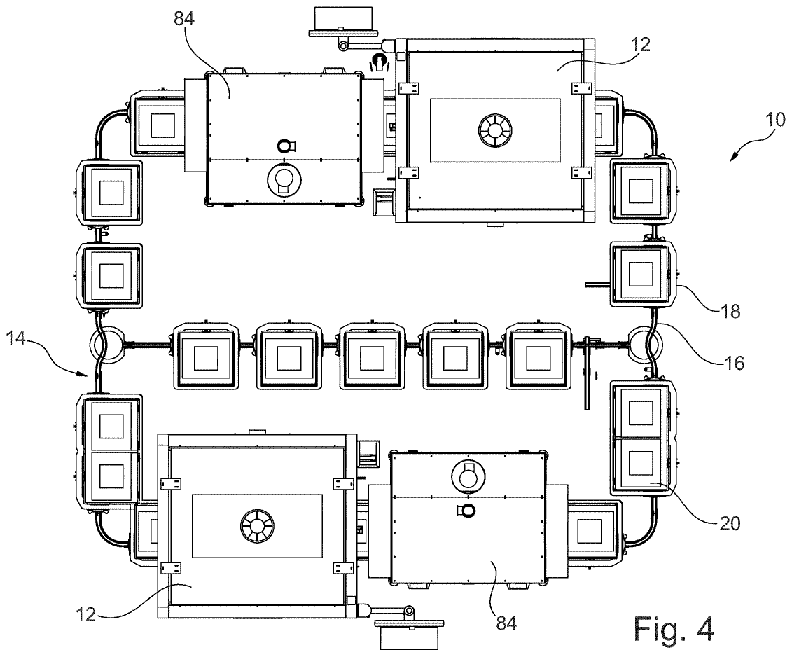

FIG. 4 a plan view of the apparatus of FIG. 3,

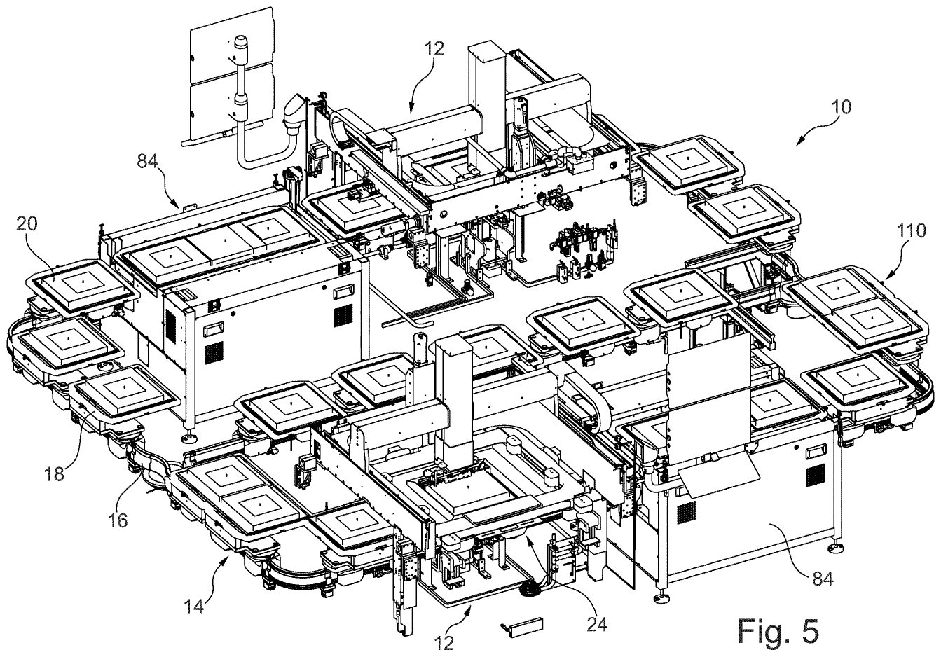

FIG. 5 a perspective view of an apparatus according to the invention with open representation of the printing and drying devices without housing

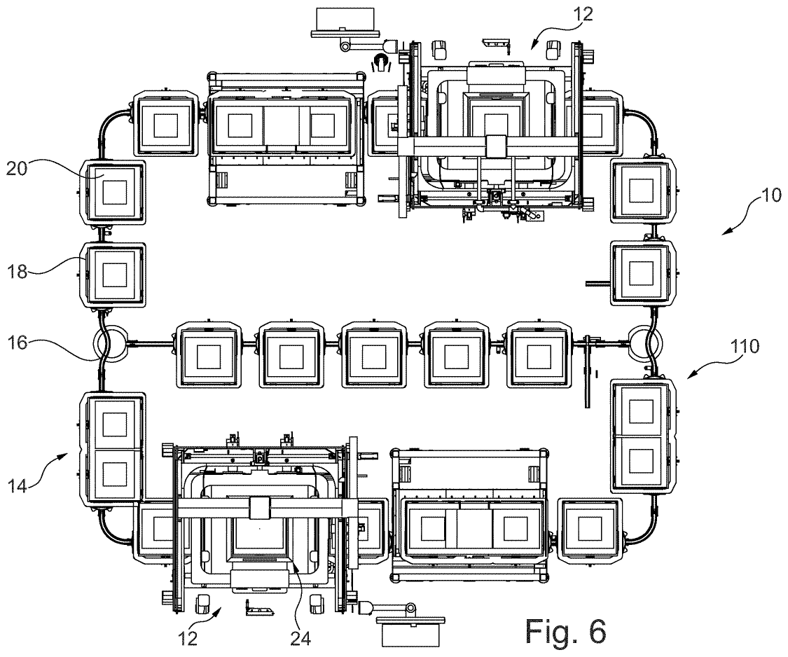

FIG. 6 a plan view of the apparatus of FIG. 5,

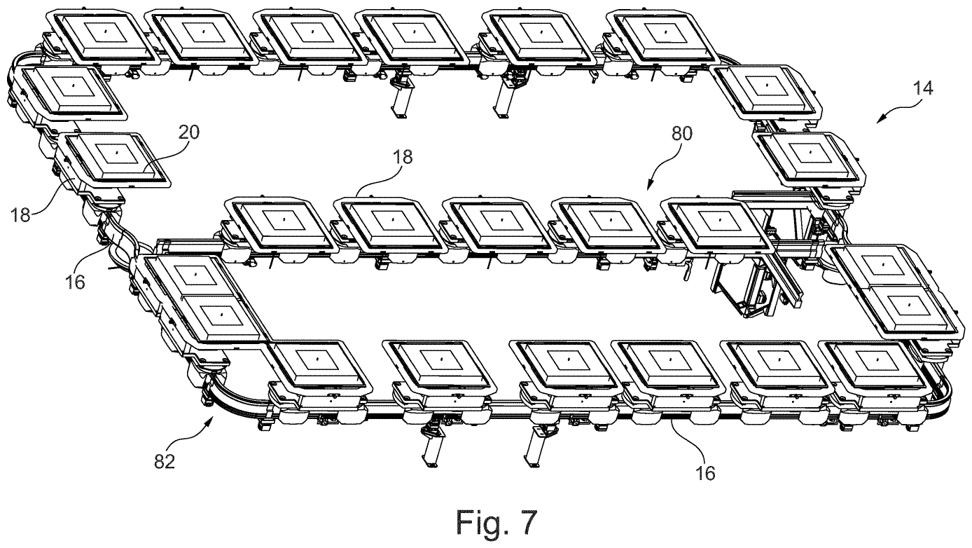

FIG. 7 a perspective view of a transport device of the apparatus according to FIGS. 1 to 6,

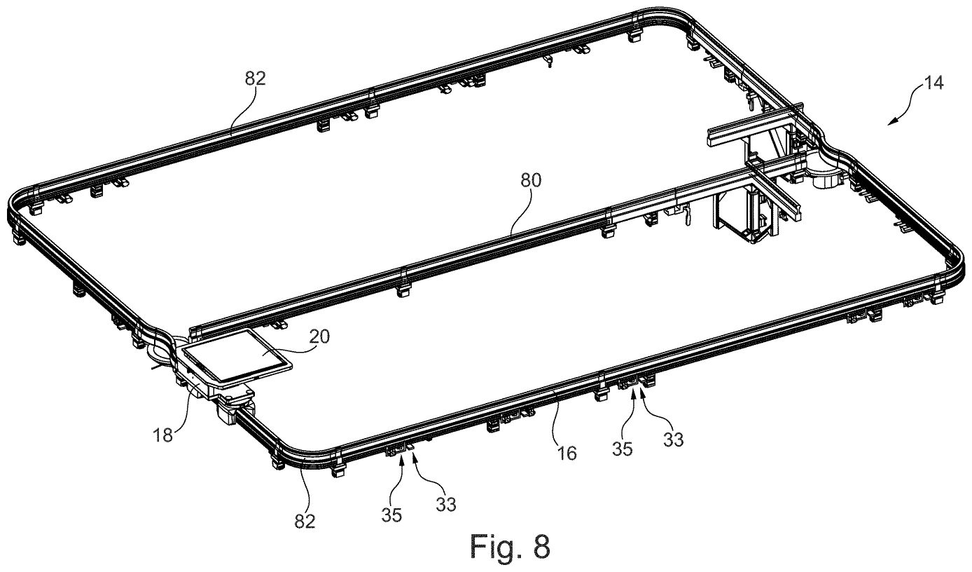

FIG. 8 a perspective view of a transport device of the apparatus according to FIGS. 1 to 6,

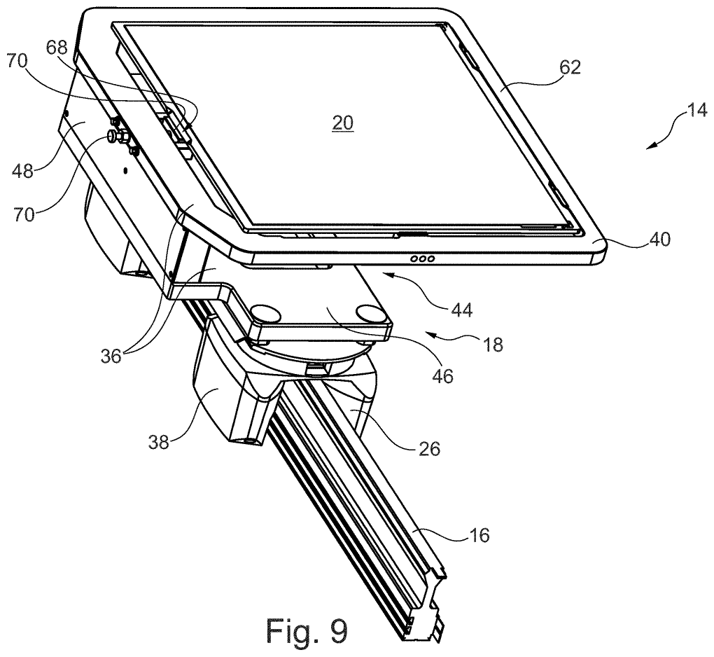

FIG. 9 an upper perspective view of a transport device with a conveying vehicle and a transport rail shown in sections,

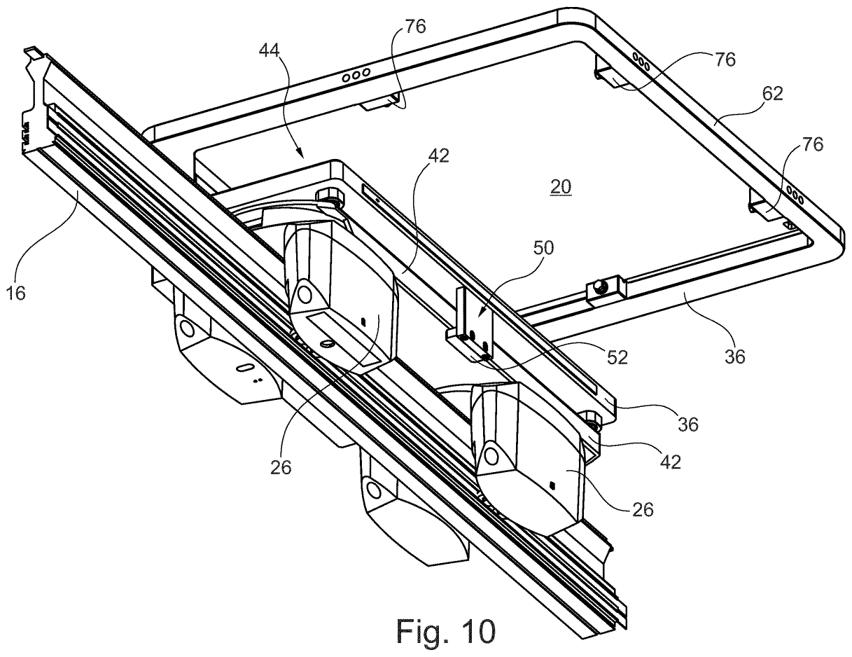

FIG. 10 a lower perspective view of a transport device with a conveying vehicle and a transport rail shown in sections,

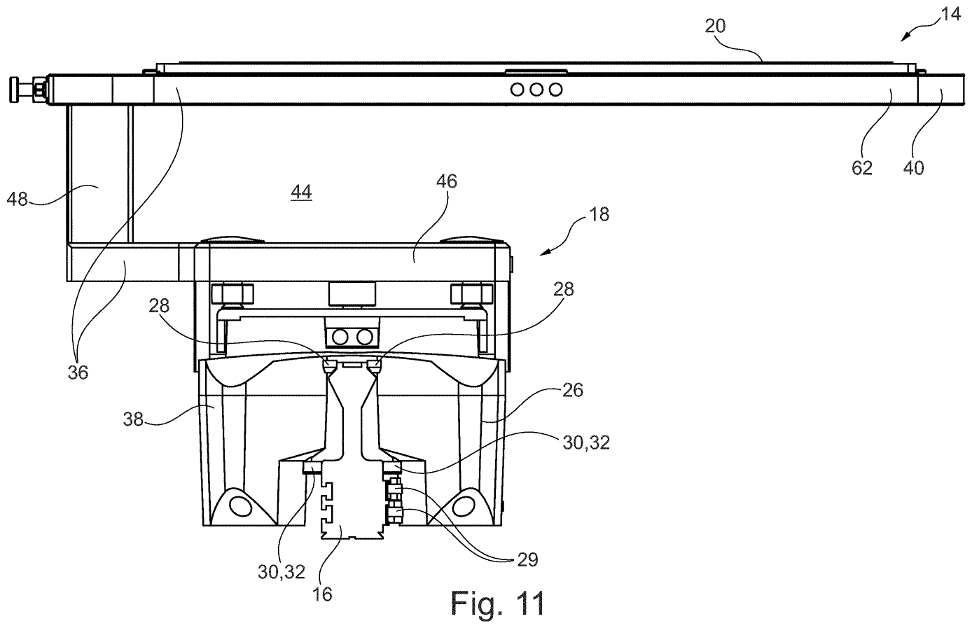

FIG. 11 a side view of the transport device according to FIGS. 9 and 10 along a longitudinal direction of the transport rail,

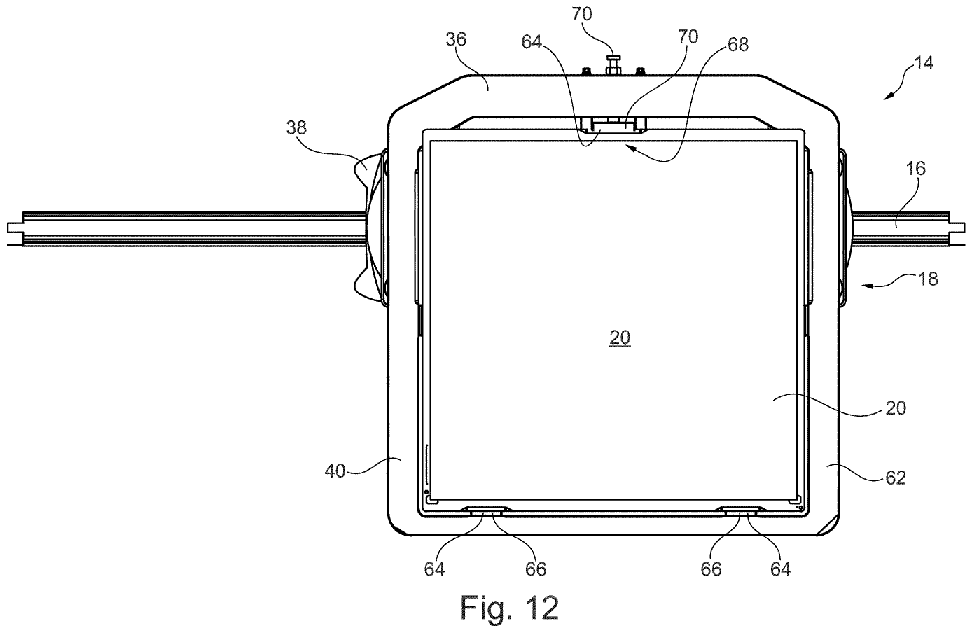

FIG. 12 a plan view of the transport device according to FIGS. 9 and 10,

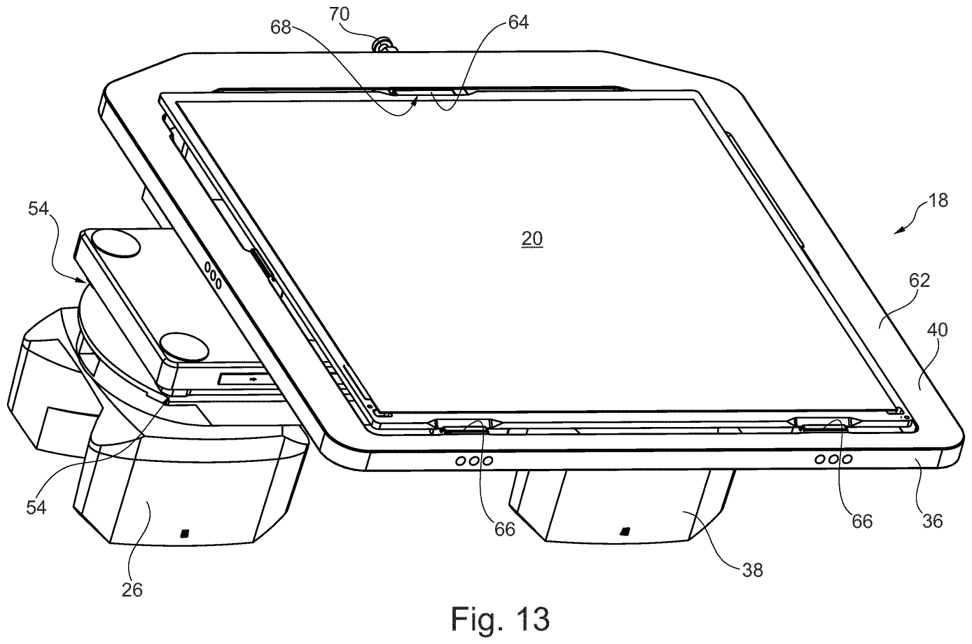

FIG. 13 a perspective view of a conveyor vehicle according to an embodiment with a workpiece carrier received therein,

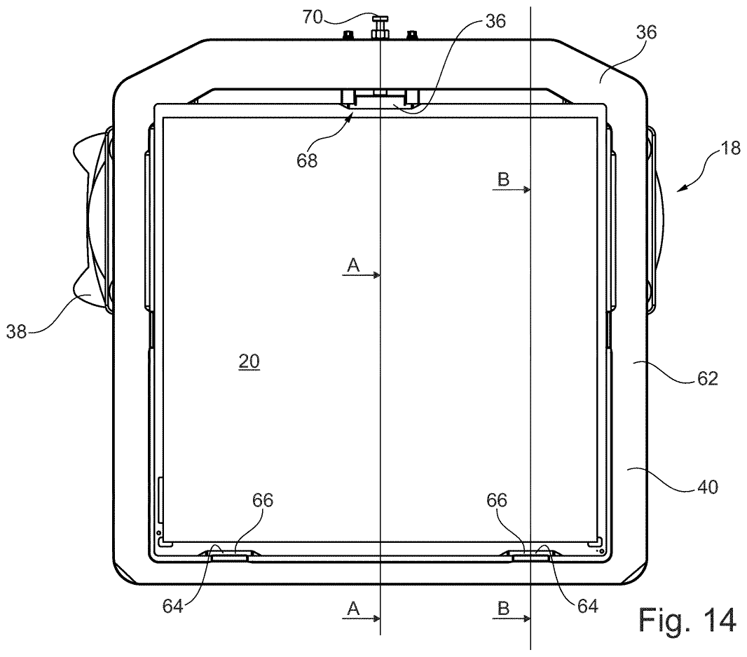

FIG. 14 a plan view of a conveyor vehicle according to FIG. 13,

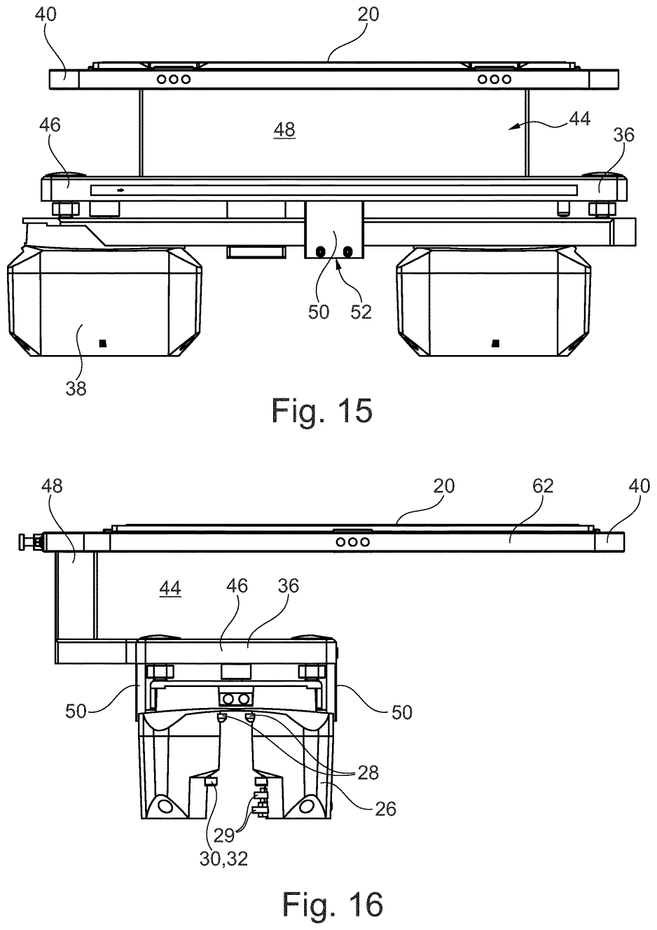

FIG. 15 a side view of a conveyor vehicle according to FIGS. 13 and 14 in a direction transverse to the transport direction,

FIG. 16 a side view of a conveyor vehicle according to FIGS. 13 and 14 in the transport direction,

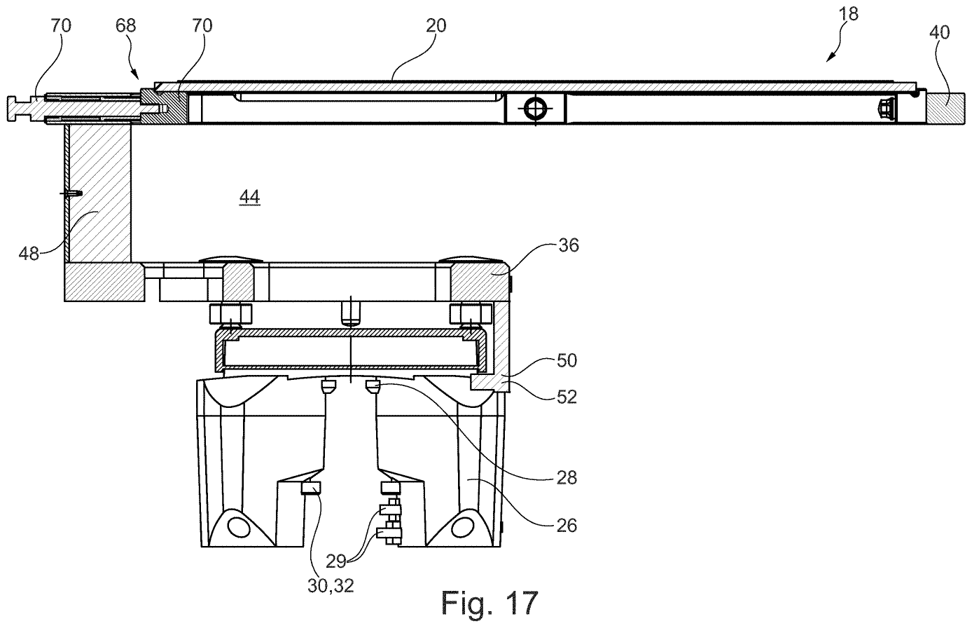

FIG. 17 a sectional illustration of the conveyor vehicle along the section line A-A from FIG. 14,

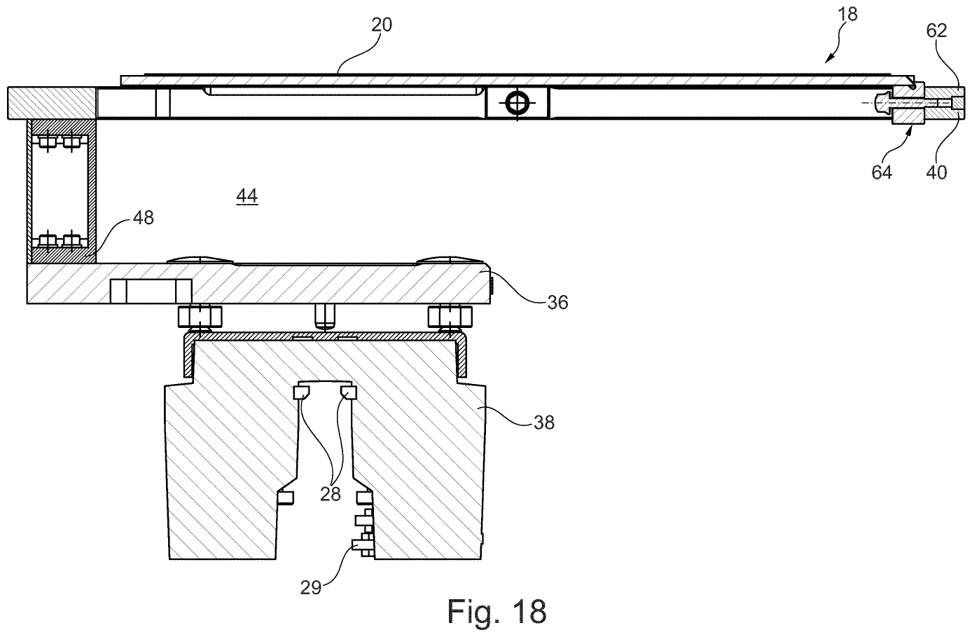

FIG. 18 a sectional illustration of the conveyor vehicle along the section line B-B from FIG. 14,

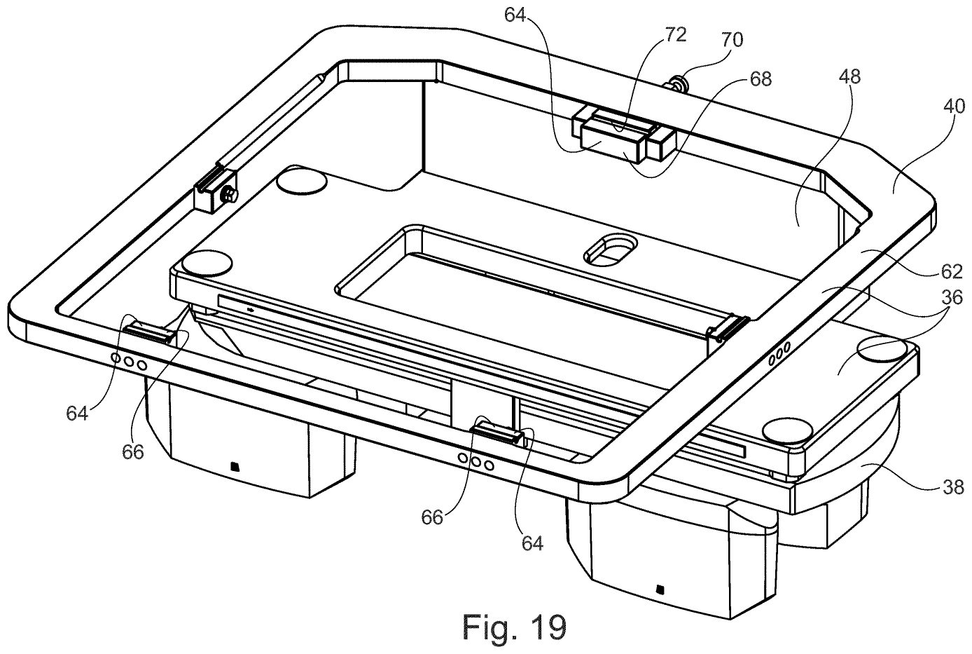

FIG. 19 an upper perspective view of a conveyor vehicle according to an embodiment without a workpiece carrier received therein,

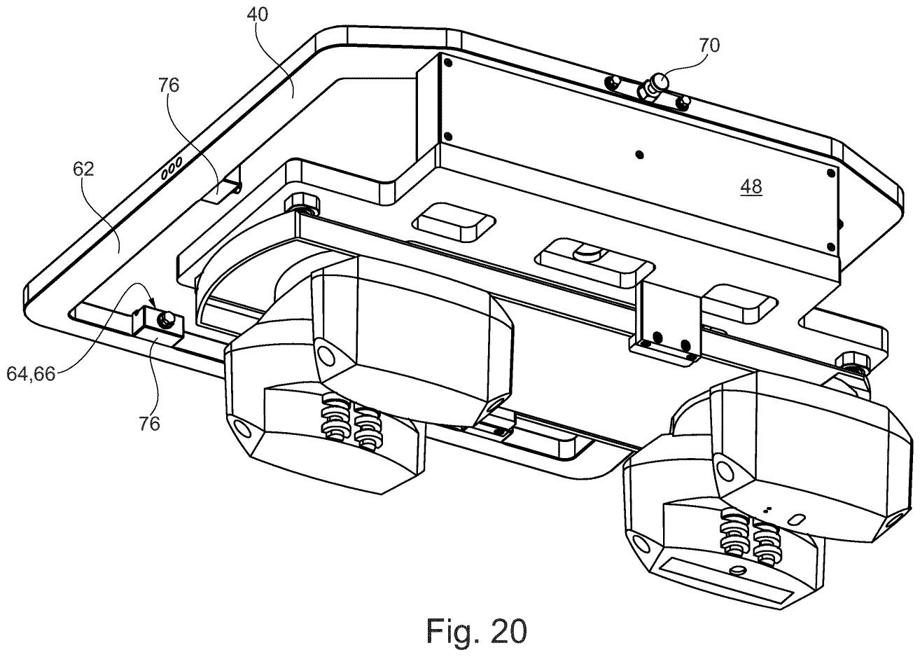

FIG. 20 a lower perspective view of a conveyor vehicle according to an embodiment without a workpiece carrier received therein,

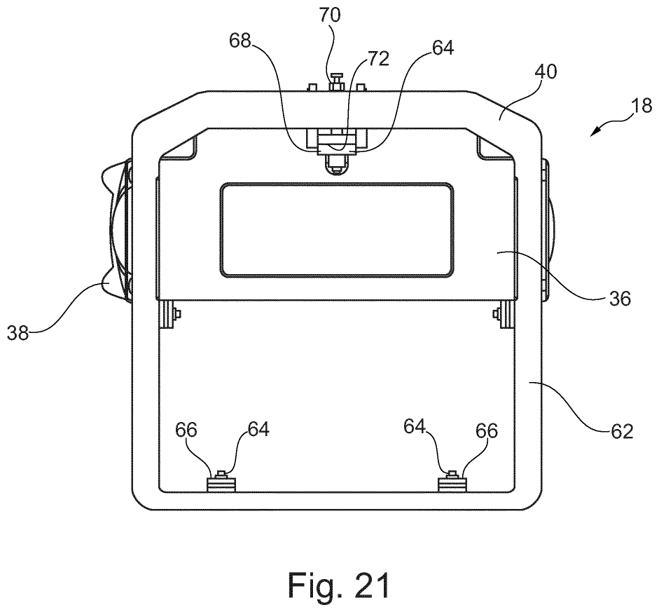

FIG. 21 a plan view of the conveyor vehicle according to FIGS. 19 and 20 without a workpiece carrier received therein,

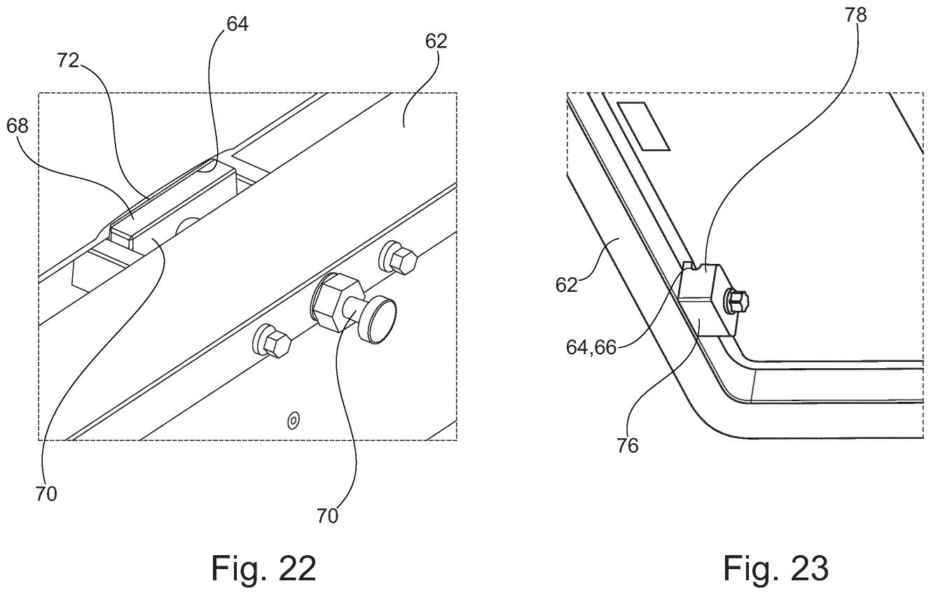

FIG. 22 a perspective detail view of a locking mechanism of a workpiece carrier mounting according to an embodiment,

FIG. 23 a perspective detail view of a fixed mounting of a workpiece carrier mounting from an underside according to an embodiment,

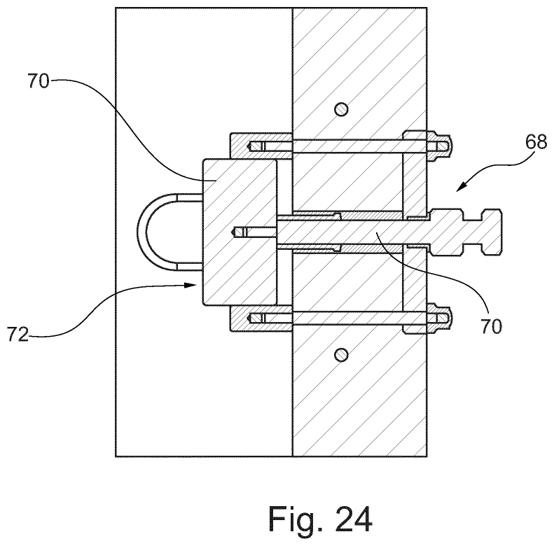

FIG. 24 a sectional illustration of a locking mechanism of a workpiece carrier mounting according to FIG. 22,

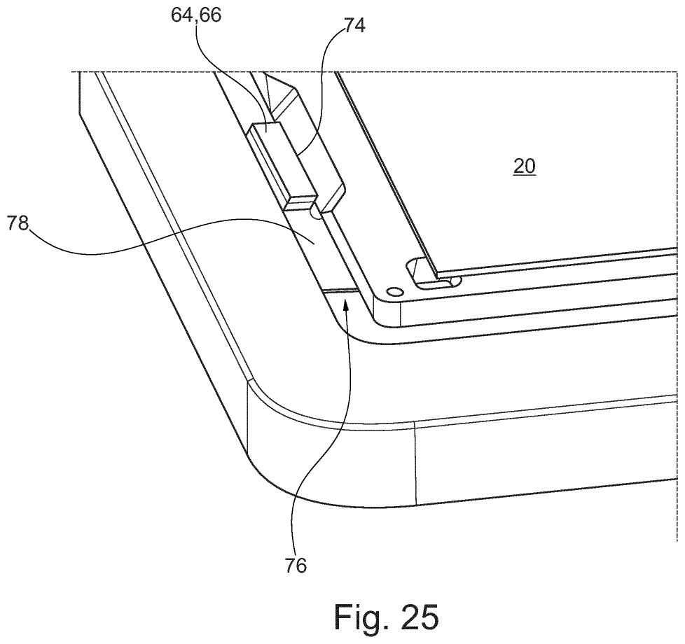

FIG. 25 a perspective detail view of a fixed mounting of a workpiece carrier mounting from an upper side according to FIG. 23,

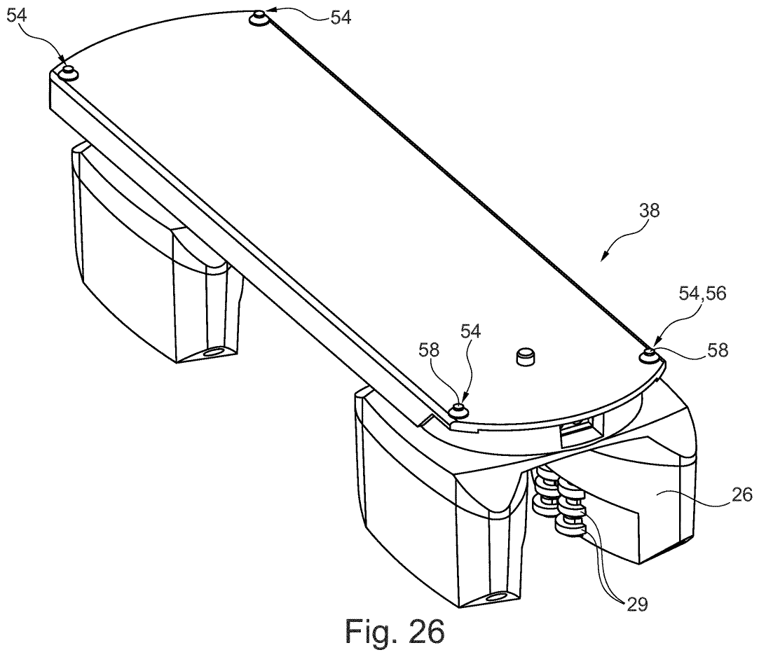

FIG. 26 an upper perspective view of a runner device according to an embodiment,

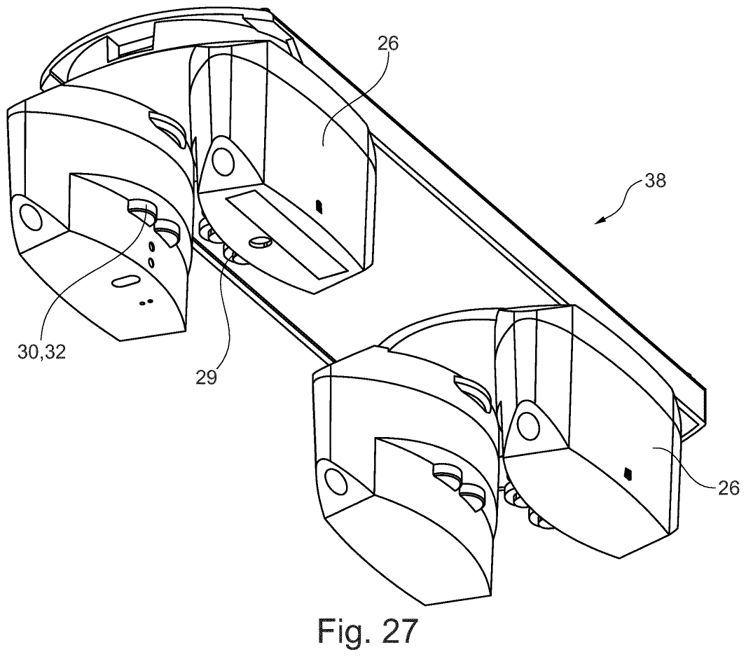

FIG. 27 a lower perspective view of a runner device according to an embodiment,

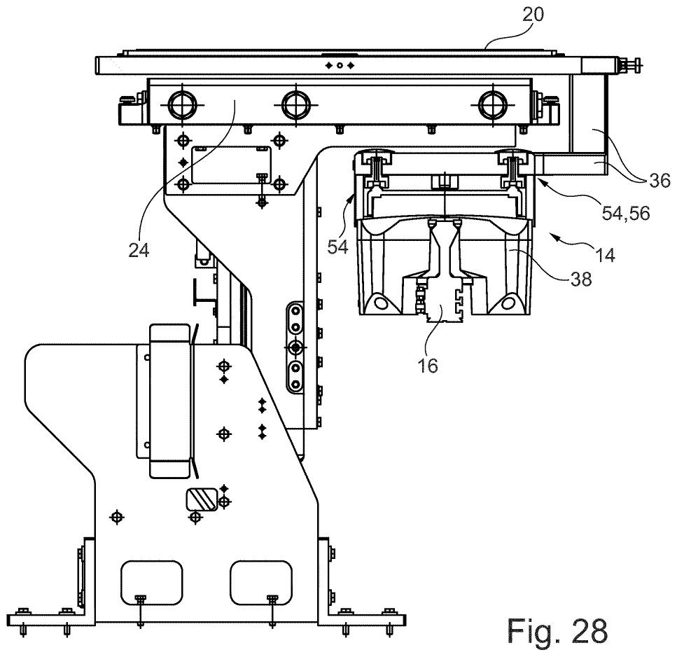

FIG. 28 a side view of a liftable printing table and of a conveyor vehicle according to an embodiment,

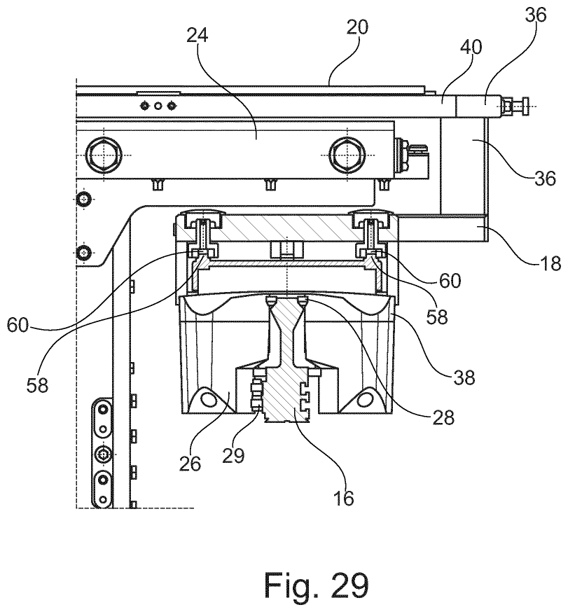

FIG. 29 a detailed view of the liftable printing table and of the conveyor vehicle according to FIG. 28,

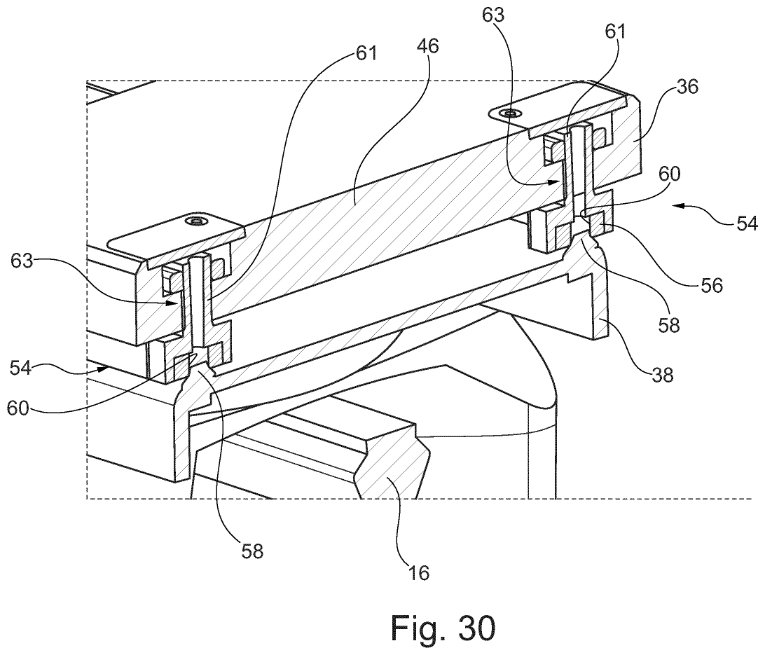

FIG. 30 a perspective sectional illustration of a multi-point mounting between the superstructure and the runner device of a conveyor vehicle according to an embodiment,

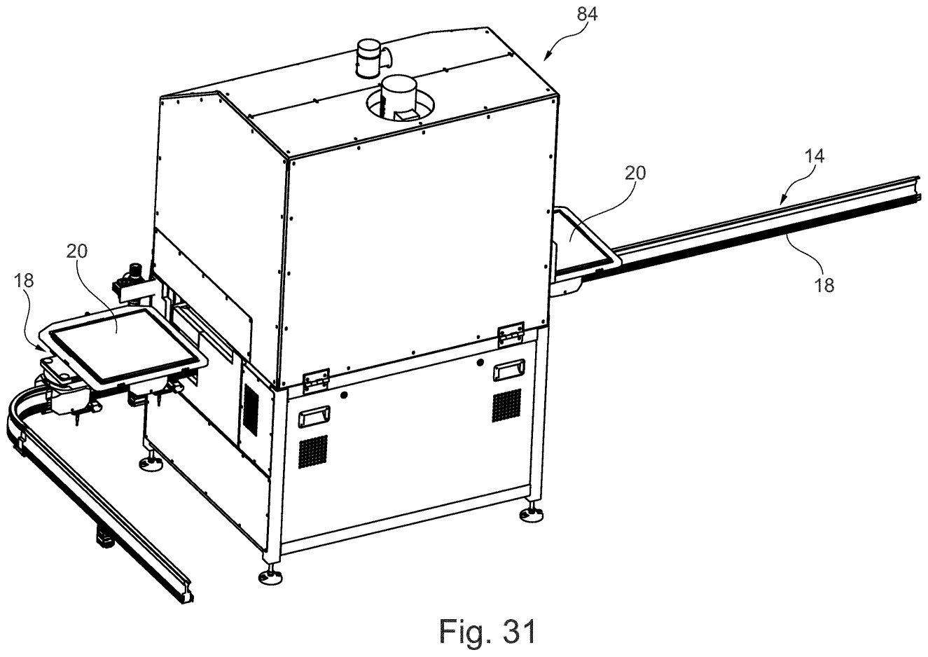

FIG. 31 a perspective view of a drying device and a sectional illustration of a transport device according to an embodiment,

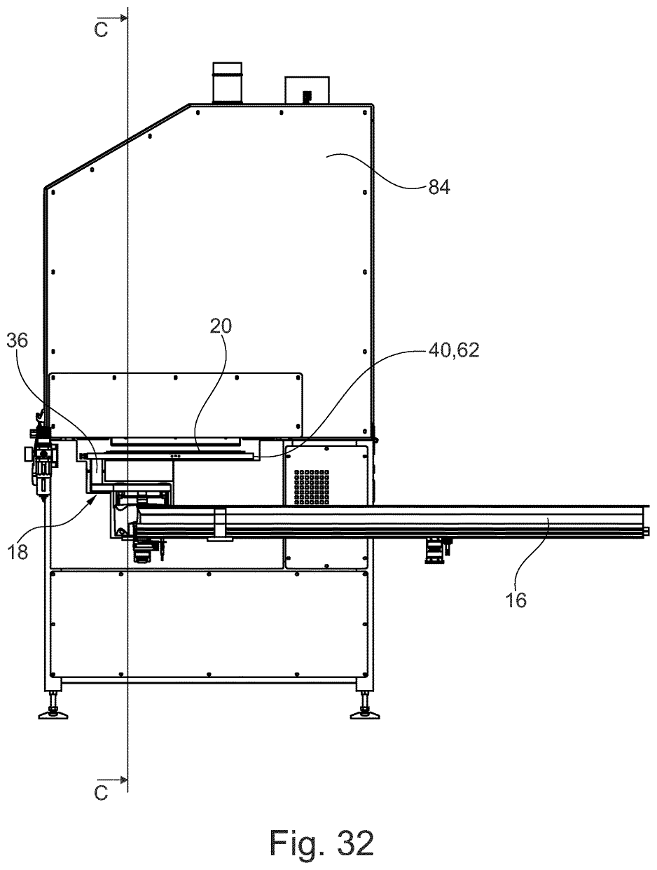

FIG. 32 a side view of the drying device according to FIG. 31 in a longitudinal direction,

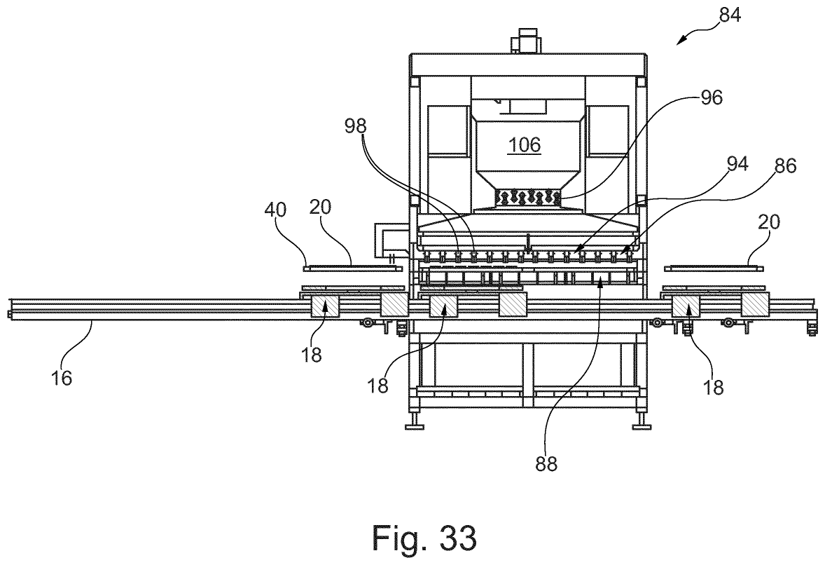

FIG. 33 a sectional illustration of the drying device along the section lines C-C from FIG. 33,

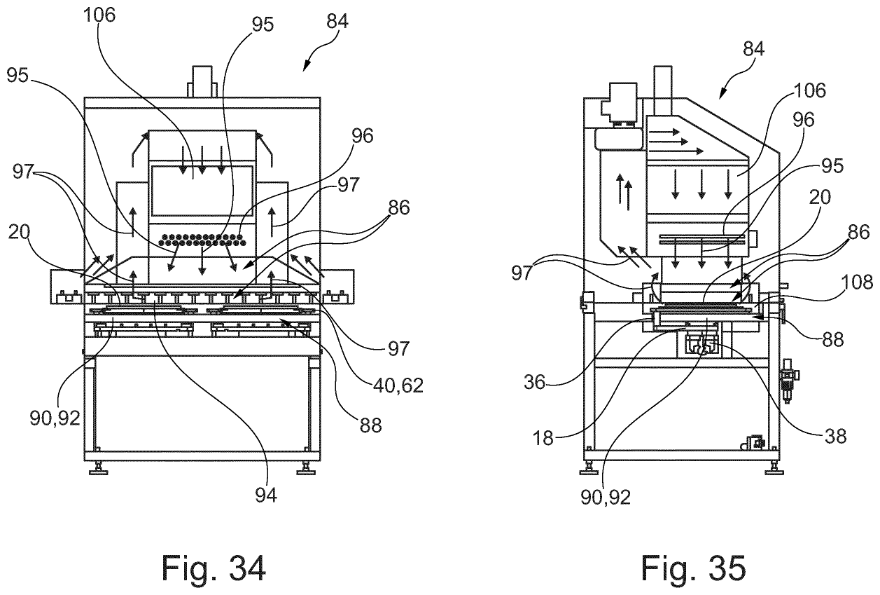

FIG. 34 a schematic longitudinal sectional illustration of the drying device according to an embodiment with illustrated air flows,

FIG. 35 a schematic cross-sectional illustration of the drying device according to an embodiment with illustrated air flows,

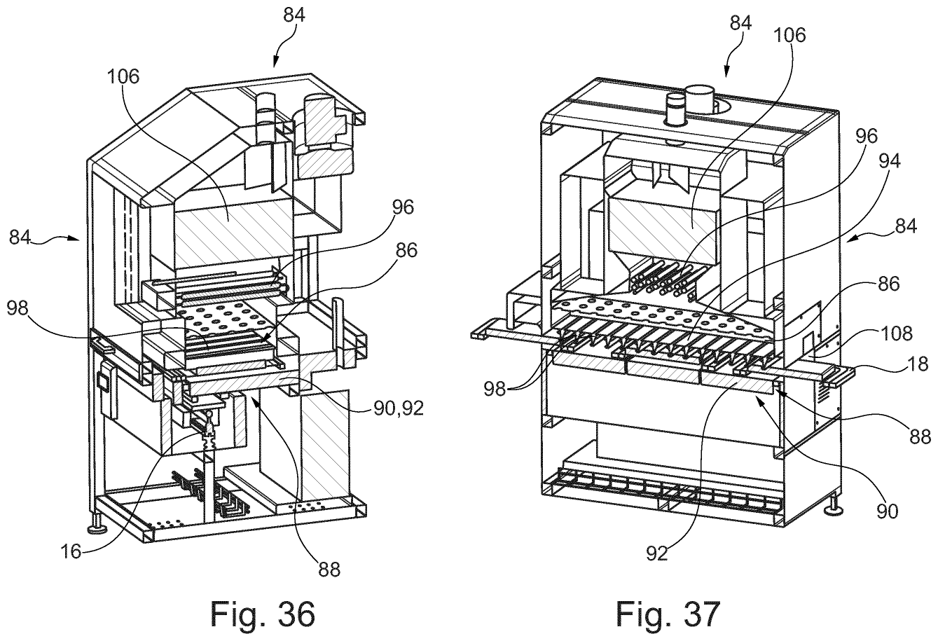

FIG. 36 a perspective cross-sectional illustration of the drying device according to an embodiment,

FIG. 37 a perspective longitudinal sectional illustration of the drying device according to an embodiment,

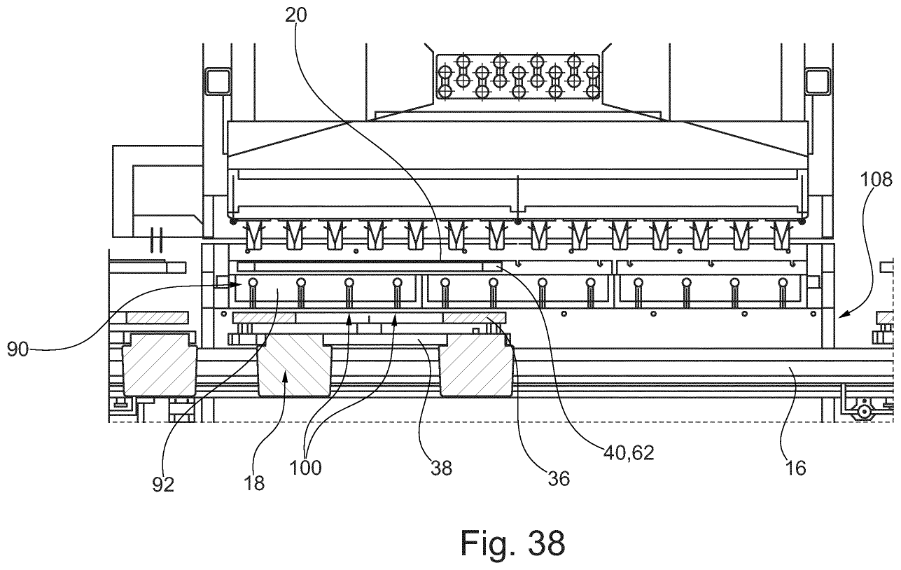

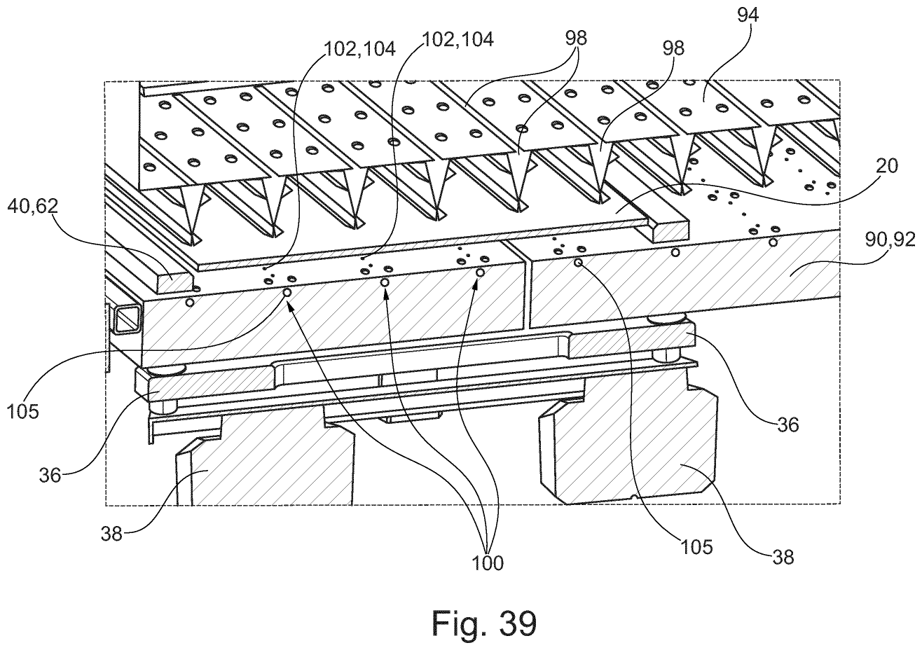

FIG. 38 a detailed view of the drying device along a longitudinal section according to an embodiment,

FIG. 39 a detailed view of the drying device in a perspective longitudinal sectional illustration according to an embodiment,

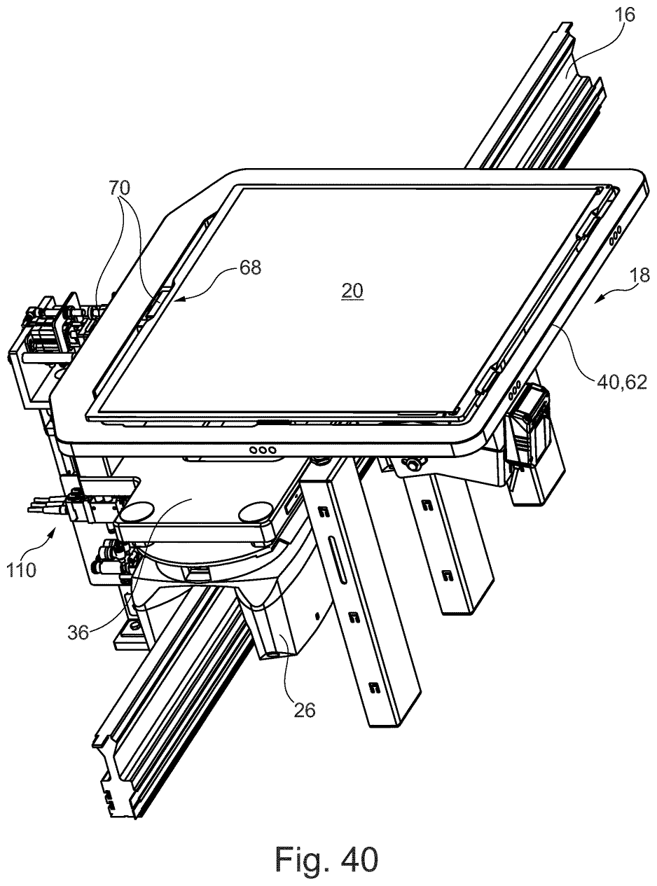

FIG. 40 a perspective view of a conveyor vehicle and a positioning and/or removal apparatus for a workpiece carrier according to an embodiment,

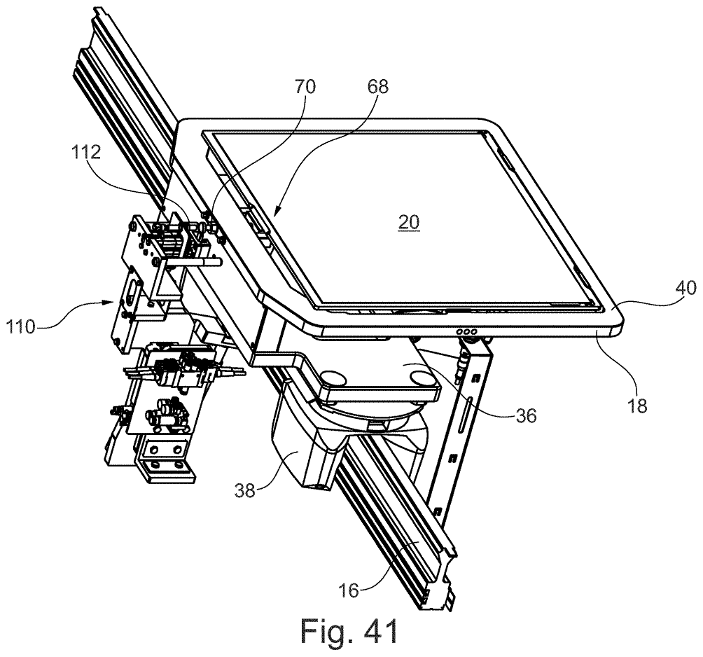

FIG. 41 a further perspective view of a conveyor vehicle and a positioning and/or removal apparatus for a workpiece carrier according to an embodiment,

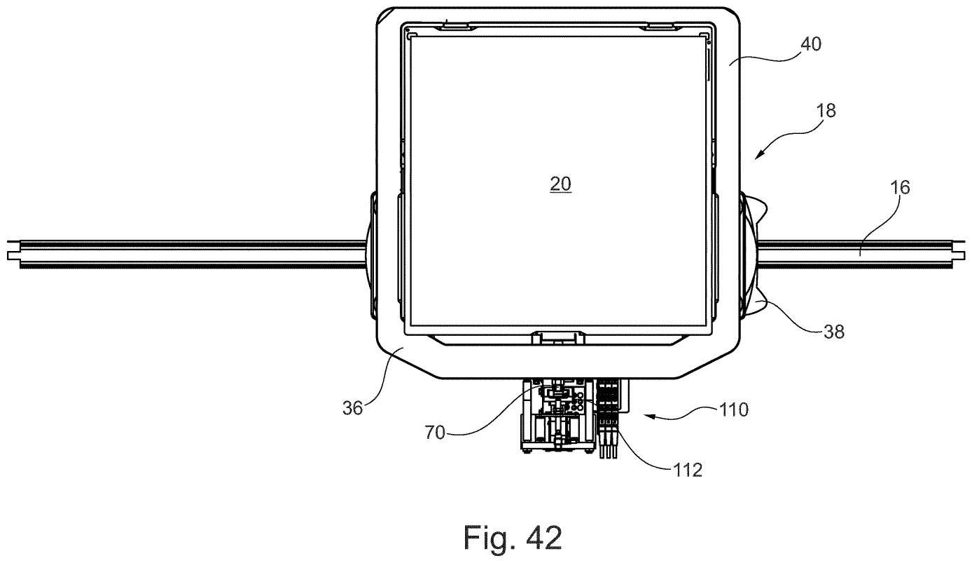

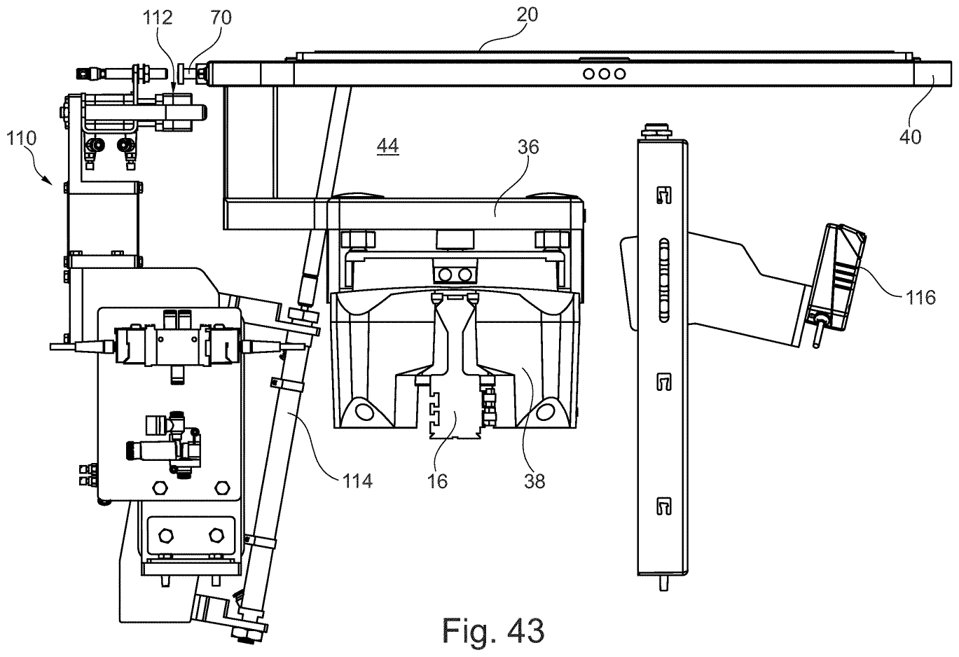

FIG. 42 a plan view of a conveyor vehicle and a positioning and/or removal apparatus for a workpiece carrier according to FIGS. 41 and 42,

FIG. 43 a side view of a conveyor vehicle and a positioning and/or removal apparatus for a workpiece carrier along a transport direction of the conveyor vehicle.

FIG. 1 shows a perspective view of an apparatus 10 for producing three-dimensional screen-printed workpieces according to an embodiment of the present invention. FIG. 2 shows a plan view of the apparatus 10 of FIG. 1. The apparatus 10 may in particular be a 3D screen-printing machine, particularly preferably a 3D screen-printing machine for producing pharmaceutical products.

The apparatus 10 has at least one printing device 12 for the layerwise production of at least one screen-printed workpiece, not illustrated in more detail here, in at least one or in a plurality of printing operations. In the embodiment according to FIGS. 1 and 2, the apparatus 10 has two printing devices 12. Furthermore, the apparatus 10 has at least one transport device 14 for the automated transport of at least one screen-printed workpiece and/or of a workpiece carrier towards and/or away from the printing device 12.

FIG. 3 shows a perspective view of the apparatus 10 according to FIG. 1, wherein, for better clarity and illustration, the transport device 14 is illustrated in the open state, in particular without housings and without frames. FIG. 4 shows a plan view of the apparatus 10 according to FIG. 3.

FIG. 5 shows a perspective view of the apparatus 10 according to FIGS. 1 to 4, wherein, for better clarity and illustration, the printing devices 12 are illustrated in the open state, in particular without housings. FIG. 6 shows a plan view of the apparatus 10 according to FIG. 5.

FIGS. 7 and 8 each illustrate a perspective view of the transport device 14 of the apparatus 10 according to FIGS. 1 to 6.

The transport device 14 can have at least one transport rail 16 and a conveying vehicle 18, arranged movably on the transport rail 16, for at least one screen-printed workpiece, not illustrated in more detail here, and/or at least one workpiece carrier 20.

The transport device 14 can be equipped with a housing 22 or with a housing 22 running along the transport device 14, as illustrated in more detail in FIGS. 1 and 2. The housing 22 can have an upper-side cover and/or a lateral cover or cladding. The risk of particle contamination on screen-printed workpieces which are moved by means of the transport device 14 can be reduced by means of such a housing 22.

The transport device 14 can furthermore preferably be set up for the automated transport of at least one screen-printed workpiece and/or of a workpiece carrier 20 towards and/or away from a printing table 24 of the printing device 12. The transport device 14 can furthermore be formed separately from the printing device 12 and/or run through the printing device 12.

A separate embodiment of the transport device 14 can be gathered in particular from FIGS. 7 and 8. Consequently, the transport device 14 can form a device which functions independently of the printing device 12, but which, despite an independent configuration, interacts with the printing device 12 or is arranged for interacting with the printing device 12, in particular in order to convey screen-printed workpieces and/or workpiece carriers 20 towards and/or away from a printing table 24 of the printing device 12.

FIGS. 9 to 25 illustrate more detailed views and details of the transport device 14 or of a conveying vehicle 18 of the transport device 14 according to an exemplary embodiment of an invention.

FIG. 9 shows an upper perspective view of a conveying vehicle 18 on a section of a transport rail 16 and FIG. 10 shows a lower perspective view of a conveying vehicle 18 on a section of a transport rail 16. FIG. 11 shows a side view of the conveying vehicle 18 according to FIGS. 9 and 10 along a longitudinal extent of the transport rail 16 and FIG. 12 shows a plan view of the conveying vehicle 18 according to FIGS. 9 to 11. FIGS. 13 to 18 show further views of the conveying vehicle 18 without representation of the transport rail 16.

FIGS. 9 to 18 show the respective conveying vehicle 18 with a workpiece carrier 20 received therein. Further views of conveying vehicle 18 without a workpiece carrier 20 received therein are illustrated in FIGS. 19 to 21.

A conveying vehicle 18 according to the embodiments illustrated in FIGS. 9 to 21 can have at least one drive device 26, which is arranged on the conveying vehicle 18 so as to travel therewith. The drive device 26 can have in particular a drive roller 28 rolling on the transport rail 16 and/or a drive motor, not illustrated in more detail here, for a drive roller 28. Furthermore, the drive device 26 can have at least one current-carrying contact roller 29, in particular a plurality of current-carrying contact rollers 29, via which an electric current can be directed from the transport rail 16 to the drive device 26, in particular for the purpose of supplying current to a drive motor.

Furthermore, the conveying vehicle 18 can have at least one supporting device 30 for supporting on the transport rail. The supporting device 30 can be in particular a supporting roller 32 rolling on the transport rail 16 for supporting the conveying vehicle 18. In particular, a conveying vehicle 18 can be equipped with supporting rollers 32 on both sides of the respective transport rail 16. It is likewise possible, instead of supporting rollers 32, to arrange sliding elements for providing a supporting function on one side or on both sides of the transport rail.

The conveying vehicle 18 can be supported on the transport rail 16 via roller contact points, in particular exclusively via roller contact points of a drive roller 28 and/or of a supporting roller 32 or via roller contact points of a plurality of drive rollers 28 and/or supporting rollers 32.

The transport rail 16 can furthermore be designed as a monorail, as can be comprehended from FIGS. 8 to 12. In particular, the transport rail 16 can have a plurality of monorail sections connected to one another, preferably monorail sections connected to one another in the longitudinal direction. Such monorail sections can, as shown in FIGS. 7 and 8, be assembled to form a transport or rail layout.

The transport rail 16 can be assembled in a modular manner or have a plurality of rail modules.

A conveying vehicle 18 according to the present invention can preferably be arranged on the transport rail 16 so as to engage around it on multiple sides, in particular so as to engage around it on three sides, as can be comprehended for example from the illustration in FIG. 11.

Furthermore, the conveying vehicle 18 can have at least one position sensor, not illustrated in more detail here, and/or one distance sensor, not illustrated in more detail here. The conveying vehicle 18 can be designed for communication with a control module 33 arranged on the transport rail 16.

A control module 33 arranged on the transport rail 16 can be designed in particular for communication with a superordinate apparatus controller 34 and/or for communication with at least one conveying vehicle 18 for controlling the transport path and/or the transport speed and/or for stopping the respective conveying vehicle 18.

In particular, a plurality of control modules 33 can be arranged at fixed positions on the transport rail 16. Such a control module 33 can be designed as an optoelectronic communication module for data exchange for the communication between the conveying vehicle 18 and the apparatus controller 34. The apparatus controller 34, in particular in the form of a so-called line controller, can be designed for controlling and/or regulating all processes and/or sequences within the apparatus 10.

At least one control unit 35, in particular a control unit 35 designed as a control cam, for controlling the speed of a conveying vehicle 18 can be arranged on the transport rail 16. Furthermore, at least one position sensor and/or distance sensor, not illustrated in more detail here, can be arranged on the transport rail 18 and/or adjacent or adjacent to the transport rail 18.

In particular, a plurality of control units 35, preferably in the form of control cams, can be arranged at fixed positions on the transport rail 16. The control units 35, in particular control cams, can preferably be designed as different control unit types or cam types.

A first control unit type or cam type can be designed to decelerate and/or stop a conveying vehicle 18 when it is driven past and/or when it is contacted by a conveying vehicle 18, in particular to decelerate it when it is driven past and/or when it is contacted at a positive flank and/or to stop it when it is driven past and/or when it is contacted at a negative flank. A deceleration can in particular be carried out to a speed of less than 5 m/min, less than 4 m/min, less than 3 m/min, less than 2.5 m/min, less than 2 m/min. A deceleration to such a low speed ensures that a further movement of the conveying vehicle 18 can take place with a reduced risk of collision. A secure further deceleration to the standstill of the conveying vehicle 18 can likewise take place as a result.

A second control unit type or cam type can be designed to accelerate a conveying vehicle 18 to a first predetermined speed when it is driven past and/or when it is contacted by a conveying vehicle 18 if the conveying vehicle 18 approaches and/or drives past at a second predetermined speed which is lower than the first predetermined speed. In this way, an acceleration of the conveying vehicle 18 to a desired first predetermined speed can be carried out with only little effort and with high operational reliability.

Such a second control unit type or cam type can furthermore be designed to accelerate and/or decelerate a conveying vehicle 18 to a second predetermined speed when it is driven past and/or when it is contacted by a conveying vehicle 18 if the conveying vehicle 18 does not approach and/or drive past at this second predetermined speed. In this way, an acceleration and/or deceleration of the conveying vehicle 18 to a desired second predetermined speed can be carried out with only little effort and with high operational reliability.

The first predetermined speed can be in particular a speed of at least 10 m/min, preferably of at least 15 m/min, preferably of at least 20 m/min, preferably of at least 25 m/min, preferably of at least 30 m/min, preferably of at least 35 m/min, preferably of at least 40 m/min, preferably of at least 45 m/min, further preferably of at least 50 m/min, even further preferably of at least 55 m/min or approximately 55 m/min. As a result, screen-printed workpieces and/or workpiece carriers can be conveyed between different positions or different devices within the apparatus with only little expenditure of time.

Furthermore, the first predetermined speed can be in particular a speed of up to 100 m/min, preferably of up to 90 m/min, preferably of up to 80 m/min, preferably of up to 70 m/min, preferably of up to 65 m/min, further preferably of up to 60 m/min, even further preferably of up to 55 m/min. A high level of transport reliability for screen-printed workpieces and/or workpiece carriers can be ensured as a result. In particular, the risk of a screen-printed workpiece and/or workpiece carrier falling down from the conveying vehicle on account of excessively high transport speeds can be reduced as a result.

The first predetermined speed can even further preferably be a speed of between 10 m/min and 100 m/min, preferably between 20 m/min and 90 m/min, preferably between 30 m/min and 80 m/min, preferably between 35 m/min and 75 m/min, preferably between 40 m/min and 75 m/min, preferably between 40 m/min and 70 m/min, preferably between 45 m/min and 65 m/min, further preferably between 50 m/min and 60 m/min, even further preferably a speed of 55 m/min. A sufficiently high productivity of the apparatus with simultaneously high operational reliability can be ensured with such a speed.

The second predetermined speed can be in particular a speed of at least 5 m/min, preferably of at least 10 m/min, preferably of at least 15 m/min, preferably of at least 20 m/min, further preferably of at least 25 m/min, even further preferably of at least 30 m/min or approximately 30 m/min. Such a speed ensures a relatively time-saving conveyance within the apparatus. At the same time, a transport speed which is reduced in comparison with a first predetermined speed can be realized as a result, for example for the purpose of further controlled deceleration.

Further preferably, the second predetermined speed can be in particular a speed of up to 70 m/min, preferably of up to 65 m/min, preferably of up to 60 m/min, preferably of up to 55 m/min, preferably of up to 50 m/min, preferably of up to 45 m/min, preferably of up to 40 m/min, further preferably of up to 35 m/min, even further preferably of up to 30 m/min. Such a speed enables a particularly easily controllable deceleration to a lower speed or a particularly easily controllable acceleration to a higher speed.

The second predetermined speed can furthermore be a speed between 5 m/min and 70 m/min, preferably between 10 m/min and 65 m/min, preferably between 15 m/min and 60 m/min, preferably between 15 m/min and 55 m/min, preferably between 15 m/min and 50 m/min, preferably between 15 m/min and 45 m/min, preferably between 20 m/min and 40 m/min, further preferably between 25 m/min and 35 m/min, even further preferably a speed of 30 m/min. Such a speed can on the one hand ensure a sufficiently high productivity within the apparatus and additionally ensure an easily controllable speed change to a higher and/or lower speed.

A third control unit type or cam type can be designed to accelerate and/or decelerate a conveying vehicle 18 to a third predetermined speed when it is driven past and/or when it is contacted, independently of the speed at which the conveying vehicle 18 approaches and/or drives past. As a result, a speed change can be brought about with only little effort and with a high degree of safety, namely to a third predetermined speed.

The third predetermined speed can be in particular a speed of at least 3 m/min, preferably of at least 5 m/min, preferably of at least 6 m/min, preferably of at least 7 m/min, preferably of at least 8 m/min, preferably of at least 10 m/min, even further preferably of at least 12 m/min or approximately 12 m/min. Such a minimum speed ensures a suitable initial speed for a deceleration to a complete standstill and/or for an acceleration to a higher speed.

The third predetermined speed can furthermore be a speed of up to 30 m/min, preferably of up to 25 m/min, preferably of up to 22 m/min, preferably of up to 20 m/min, preferably of up to 18 m/min, preferably of up to 16 m/min, preferably of up to 14 m/min, even further preferably of up to 12 m/min. Such a maximum speed ensures, with a high degree of safety, a suitable initial speed for a deceleration and/or for an acceleration to a higher speed.

The third predetermined speed can furthermore preferably be a speed between 3 m/min and 30 m/min, preferably between 5 m/min and 25 m/min, preferably between 7 m/min and 22 m/min, preferably between 8 m/min and 20 m/min, preferably between 8 m/min and 18 m/min, preferably between 8 m/min and 16 m/min, preferably between 10 m/min and 14 m/min, even further preferably a speed of 12 m/min. Such a speed is particularly suitable as an initial speed for a complete deceleration to a standstill of the conveying vehicle and also for an acceleration to a higher speed, with at the same time a low risk of collisions or a falling down of a screen-printed workpiece or of a workpiece carrier from the conveying vehicle.

The control units 35 and/or control cams can be designed in a switchable and non-switchable embodiment. Switchable control units 35 and/or control cams can be operated without current—free of electric current.

The transport rail 16 can have predefined stopping points for a conveying vehicle 18, at which preferably at least one control module 33 and/or one control unit 35 is provided. At least one control module 33 and/or at least one control unit 35 and/or the apparatus controller 34 can be designed to stop a conveying vehicle 18 at a predefined stopping point only if required. Furthermore, at least one sensor can be provided at a predefined stopping point, which detects when a conveying vehicle has driven to a control module 33, independently of whether the conveying vehicle 18 has stopped at this control module 33 or not. Such a sensor can be designed as a control module 33 or as part of a control module 33.

The transport device 14 can furthermore have a collision monitoring system for avoiding collisions between conveying vehicles 18, wherein the collision monitoring system is preferably set up for signal processing of distance and/or position sensors and/or of control modules 33 and/or of control units 35 of the transport rail 16.

As can furthermore be comprehended from FIGS. 9 to 21, the conveying vehicle 18 can have a superstructure 36 for receiving a workpiece carrier 20 for screen-printed workpieces. Likewise, the conveying vehicle 18 can have a runner device 38 having the drive device 26, wherein the superstructure 36 is preferably arranged on the runner device 38 of the conveying vehicle 18. Separate views of the runner device 38 can be comprehended from FIGS. 26 and 27.

The superstructure 36 and/or a carrying section 40 of the superstructure 36 can preferably define a receiving plane for a workpiece carrier 20, which extends at a distance from the runner device 38 and/or from the drive device 26, in particular extends at a vertical distance.

The superstructure 36, in particular with a workpiece carrier 20 received therein, in a plan view, as illustrated in FIG. 12, can cover the runner device 38 and/or the drive device 26 at least in sections and/or project beyond at least one side end 42 of the runner device 38.

Overall, the superstructure 36 can be C-shaped. The C-shaped formation can be comprehended for example from the illustration in FIG. 11. Furthermore, the superstructure 36 can form or delimit a free space 44 for receiving a printing table 24 of the printing device 12.

In a cross-section of the superstructure 36, the surface center of the superstructure 36 can be arranged laterally offset with respect to the center of gravity of the superstructure 36. The surface center of the cross-section of the superstructure 36 can be formed in particular from the averaging of all points of the cross-sectional surfaces of the base section 46, of the connecting section 48 and of the carrying section 40. In a cross-section of the superstructure 36, the surface center of the superstructure 36 can be arranged in particular laterally offset or offset in a direction transversely to the longitudinal extent of the transport rail 16. Such a cross-section of the superstructure 36 can be comprehended in particular from FIGS. 17 and 18 and advantageously enables the arrangement of a printing table 24 of a printing device 12 within the free space 44 for the purpose of contacting the underside of the respectively received workpiece carrier 20.

The superstructure 36 can have a base section 46 for fastening to the runner device 38, a carrying section 40 for receiving a workpiece carrier 20 and a connecting section 48 for connecting the carrying section 40 to the base section 46. In this case, the free space 44 for receiving a printing table 24 and/or for contacting the underside of a workpiece carrier 20 can preferably be formed between the base section 46 and the carrying section 40 and/or can be delimited laterally by the connecting section 48.

The superstructure 36 and/or the carrying section 40 of the superstructure 36 can be mounted so as to be movable relative to the runner device 38 in the vertical direction and/or can be arranged so as to be adjustable in height, as illustrated in detail in FIGS. 28 to 30. The superstructure 36 and/or the carrying section 40 of the superstructure 36 can be arranged so as to be raisable in particular relative to the runner device 38 by a printing table 24 of the printing device 12.

A stroke and/or a vertical movability of the superstructure 36 and/or of the carrying section 40 of the superstructure 36 relative to the runner device 38 can be delimited, preferably by a mechanical stroke-limiting device 50. Such a stroke-limiting device 50 can be formed in particular as a stroke-limiting claw 52, as illustrated by way of example in FIG. 10.

The stroke-limiting device 50 and/or the stroke-limiting claw 52 can preferably be arranged on the superstructure 36 and/or engage behind the runner device 38 in a form-fitting manner. By raising the superstructure 36, the stroke-limiting device 50 and/or the stroke-limiting claw 52 can be brought into form-fitting contact engagement with the runner device 38.

The superstructure 36 can furthermore be fastened to the runner device 38 and/or guided in a vertically movable manner via a multi-point bearing 54, in particular via a 4-point bearing. Such a vertical movability can enable raising without actuation of adjusting screws or without release of a locking mechanism. It can be a free vertical movability—within predefined limits. The multi-point bearing 54 can be formed in particular by a plurality of bearings 56. A bearing 56 between the superstructure 36 and the runner device 38 can be formed by a bearing mandrel 58 and a mandrel receptacle 60 for the bearing mandrel 58.

The multi-point bearing 54 and/or at least one bearing 56 between the superstructure 36 and the runner device 38 can furthermore be designed for self-centering and/or self-alignment of the superstructure 36 relative to the runner device 38. For this purpose, at least one bearing mandrel 58 can taper conically and/or at least one mandrel receptacle 60 can taper conically. The mandrel receptacle 60 can be formed in a complementary manner to the bearing mandrel 58 or with a conicity deviating from the bearing mandrel 58. Such a configuration can ensure a secure self-centering of the superstructure 36 relative to the runner device 38 with only little effort.

A bearing 56 between the superstructure 36 and the runner device 38 can furthermore be equipped with an adjusting screw 61. Such an adjusting screw 61 can be screwed into a thread 63 of the base section 46. The mandrel receptacle 60 can be formed on the adjusting screw 61, in particular on the lower end of the adjusting screw 61. By screwing in or screwing out the adjusting screw 61, a vertical adjustment of the superstructure 36 relative to the runner device can therefore be carried out. For a raising of the superstructure 36 by a printing table 24, a correct initial height can be set in this way.

The conveying vehicle 18, in particular the superstructure 36 of the conveying vehicle 18 and/or the carrying section 40 of the superstructure 36, can be designed, as illustrated by way of example in FIGS. 9 to 25, for receiving and/or fixing a workpiece carrier 20 in a form-fitting and/or force-fitting and/or material-bonded manner and/or at least in sections. Additionally or alternatively, the superstructure 36 and/or the carrying section 40 of the superstructure 36 can be designed for receiving and/or enclosing a workpiece carrier 20 in a defined and/or play-free manner.

Further preferably, the conveying vehicle 18, in particular the superstructure 36 of the conveying vehicle 18 and/or the carrying section 40 of the superstructure 36, can have, as illustrated by way of example in FIGS. 9 to 25, a frame device 62, in particular a frame device 62 for receiving and/or fixing a workpiece carrier 20 in a form-fitting and/or force-fitting and/or material-bonded manner and/or at least in sections.

It is likewise possible for the conveying vehicle 18, in particular the superstructure 36 of the conveying vehicle 18 and/or the carrying section 40 of the superstructure 36, to have, instead of a frame device 62, a carrying plate, not illustrated in more detail here, in particular a carrying plate for receiving and/or fixing a workpiece carrier 20 in a form-fitting and/or force-fitting and/or material-bonded manner and/or at least in sections. Such a carrying plate can be designed for example with a recess for the arrangement of a workpiece carrier 20.

The frame device 62 can preferably be designed for receiving and/or enclosing a workpiece carrier 20 in a defined and/or play-free manner. Likewise, the frame device 62 can preferably delimit a free space 44 for contacting the underside of a workpiece carrier 20 received and/or enclosed therein.

As illustrated in detail in FIGS. 19 to 25, the conveying vehicle 18, in particular the superstructure 36 and/or the carrying section 40 and/or the frame device 62, can have a workpiece carrier mounting 64 for fixing and/or for clamping and/or for fastening a workpiece carrier 20 in a force-fitting and/or form-fitting manner. The workpiece carrier mounting 64 can preferably be designed as a multi-point bearing, in particular as a 3-point bearing.

The workpiece carrier mounting 64 can be formed in a particularly preferred manner by at least one fixed mounting 66 and/or by at least one locking mechanism 68, in particular a plurality of fixed mountings 66 and an oppositely arranged locking mechanism 68, as can be comprehended from FIGS. 19 to 25.

The locking mechanism 68 can preferably be formed by a slide 70 with a contact surface 72 which is chamfered and/or inclined with respect to the sliding direction of the slide 70. The locking mechanism 68 can be formed in a particularly preferred manner by a slide 70 which is under spring pretension and/or as a snap closure. Furthermore, a fixed mounting 66 can also be formed with a contact surface 74 which is chamfered and/or inclined with respect to the sliding direction of the slide 70, in particular a plurality of fixed mountings 66 can in each case be formed with an inclined contact surface 74.

According to a further preferred configuration, the superstructure 36, in particular the carrying section 40 and/or the frame device 62, can have support sections 76 for contacting and/or raising the superstructure 36 by a printing table 24 of the printing device 12.

The support sections 76 can preferably be formed on an underside of the carrying section 40 and/or of the frame device 62. In particular, the support sections 76 can be formed on shaped elements 78 which are formed and/or arranged on an inner side of the frame device 62. The support sections 76 can be formed on the under-sides of the shaped elements 78. In contrast, a fixed mounting 66 and/or an inclined contact surface 74 can be formed on an upper side of the respective shaped element 78.

The support sections 76 can be formed and/or arranged for contacting by a printing table 24 in a manner load-free for the workpiece carrier mounting 64. The superstructure 36 can be contactable and/or liftable via the support sections 76 by a printing table 24 in a manner load-free for the workpiece carrier mounting 64. Such contacting in a manner load-free for the workpiece carrier mounting 64 can be ensured in particular when the support sections 76 take place by a printing table 24 before or simultaneously with the contacting of the respective workpiece carrier 20 by the printing table 24.

The printing device 12 can furthermore have a liftable printing table 24, preferably a liftable vacuum printing table. A liftable printing table 24 is illustrated by way of example in FIG. 28.

In this case, the printing table 24 of the printing device 12 can be designed for planar contacting of a workpiece carrier 20 received in a conveying vehicle 18 and/or for contacting the support sections 76 of the superstructure 36 of the conveying vehicle 18. Furthermore, the printing table 24 of the printing device 12 can be designed to raise the superstructure 36 via the support sections 76 in a manner load-free for the workpiece carrier mounting, in particular with simultaneous and/or successive contacting of the support sections 76 and of the workpiece carrier 20.

In a plan view of the transport device 14, for example according to FIG. 12, and/or in a cross-section of the transport device 14, the center of gravity of the conveying vehicle 18 can be arranged offset with respect to the transport rail 16. The center of gravity of the conveying vehicle 18 can be arranged laterally offset with respect to the transport rail 16 or laterally offset with respect to a center of gravity of the transport rail 16. In particular, the center of gravity of the conveying vehicle 18 can be arranged laterally offset with respect to the transport rail 16 in a horizontal direction and transversely to a longitudinal extent of the transport rail 16. In this case, a longitudinal extent of the transport rail 16 can predefine the transport direction of a conveying vehicle 18 on the respective transport rail 16.

Further preferably, in a plan view of the conveying vehicle 18, as illustrated for example in FIGS. 12 and 18, the center of gravity of the superstructure 36 can be arranged offset with respect to the center of gravity of the runner device 38. The center of gravity of the superstructure 36 can be arranged laterally offset with respect to the center of gravity of the runner device 38. In particular, the center of gravity of the superstructure 36 can be arranged laterally offset relative to the center of gravity of the runner device 38 in a horizontal direction and transversely to a longitudinal extent of the transport rail 16 and/or in a horizontal direction and transversely to a longitudinal extent of the conveying vehicle 18. In this case, a longitudinal extent of the transport rail 16 can again predefine the transport direction of a conveying vehicle 18 on the respective transport rail 16. In this case, a longitudinal extent of the conveying vehicle 18 can run along the transport direction of the conveying vehicle 18 on the respective transport rail 16.

An advantageous driving behavior in cornering situations of the conveying vehicle 18 can result as a result of such a weight distribution, as a result of which in particular an undesired tilting of the conveying vehicle 18 or of the superstructure 36 relative to the runner device 28 can be avoided.

The transport device 14 and/or the rail system of the transport device 14 can be formed in a preferred manner as a transport circuit, as illustrated in more detail in FIGS. 7 and 8. In this case, the rail system of the transport device 14 can have a bypass section 80. On such a bypass section 80, conveying vehicles 18 can be temporarily positioned or stopped before they are filtered into a main section 82 of the rail system of the transport device 14 again or move into such a main section 82.

The transport device 14 can be set up in particular for the automated transport of at least one screen-printed workpiece and/or of a workpiece carrier 20 in the circuit between a printing device 12 and a position spaced apart from the printing device 12. A position spaced apart from the printing device 12 can be any position outside the printing device 12 and along the rail system. Positions outside the printing device 12 can be arranged along the main section 82 or along the bypass section 80.

As shown in FIGS. 1 to 6, the apparatus 10 can have a plurality of printing devices 12. The transport device 14 can run between the printing devices 12. Accordingly, a screen-printed workpiece and/or a workpiece carrier 20 can be transported between the printing devices 12 by means of the transport device 14.

The apparatus 10 can furthermore be equipped with at least one drying device 84 for at least one screen-printed workpiece, in particular with a plurality of drying devices 84, as shown in FIGS. 1 to 6. Preferably, each printing device 12 can be assigned a drying device 84 and/or be arranged downstream of the transport device 14 in the course thereof. The at least one drying device 84 can furthermore preferably be formed for the continuous drying pass through and/or for the stationary drying.

The transport device 14 can furthermore preferably run for the transport of a screen-printed workpiece and/or of a workpiece carrier 20 towards and/or away from the drying device 84 and/or through the drying device 84. Likewise, the transport device 14 can be formed for the transport of a screen-printed workpiece and/or of a workpiece carrier 20 between the printing device 12 and the drying device 84.

In particular, the transport device 14 can run at least in sections through the drying device 84, and/or the transport device 14 can be formed separately from the drying device 84. As a result, an overall modular construction of the apparatus 10 can be made possible or further promoted. The transport device 14 can be configured independently of the drying device 84 and can be arranged for connecting the drying device 84 to at least one further device or position within the apparatus 10.

Furthermore, a transport device 14 formed separately and/or independently of the drying device 84 can simplify the supplementation and/or the replacement of the respective drying device 84, in particular the supplementation with further units within the apparatus or the replacement of the drying device 84 by another drying device 84.

Despite the transport device 14 being formed separately and/or independently of the drying device 84, it can be adapted to the drying device 84, in particular in order to enable a reliable transport of a workpiece carrier 20 and/or of a screen-printed workpiece towards and/or away from the drying device 84.

Details of such a drying device 84 are illustrated in FIGS. 31 to 39. The drying device 84 can be formed in a particularly preferred manner for the drying of at least one screen-printed workpiece and for the simultaneous cooling of a workpiece carrier 20 carrying the respective screen-printed workpiece to be dried.

The interior of the drying device 84 is illustrated in the longitudinal sectional illustrations in FIGS. 33, 34 and 37 and also in the cross-sectional illustrations in FIGS. 35 and 36. The drying device 84 can have in particular a drying section 86 for the drying of screen-printed workpieces and a cooling section 88 for the cooling of workpiece carriers 20.

For this purpose, the drying device 84 can be divided in particular into a drying section 86 and a cooling section 88, wherein at least one nozzle device 90 and/or perforated plate 92 for the supply of a cooling air flow, in particular of compressed cooling air, can preferably be arranged within the cooling section 88.

By means of a subdivision of the drying device 84 into a drying section 86 and a cooling section 88, the risk of a warm drying air flow reaching an underside of the respective workpiece carrier 20 to be cooled can be avoided or reduced. An undesired heating of the workpiece carrier 20 can therefore be prevented.

With a suitable subdivision of the drying device 84 into a drying section 86 and a cooling section 88, in particular a mixing of drying air flows and cooling air flows can be prevented or reduced, in particular temporarily prevented or reduced. Such a mixing of drying air flows and cooling air flows can be prevented or kept low in a particularly preferred manner until the respective function of the drying air flows and cooling air flows has been fulfilled, namely until a drying of the respective screen-printed workpieces takes place and a cooling of the workpiece carrier has been ensured simultaneously with the drying or at least temporarily during the drying.

A subdivision of the drying device 84 into a drying section 86 and a cooling section 88 can be promoted in particular in that a conveying vehicle 18, preferably with workpiece carrier 20 positioned thereon, is positioned within the drying device 84. The conveying vehicle 18 or a workpiece carrier 20 positioned thereon itself can advantageously contribute to the subdivision of the drying device 84 into a drying section 86 and a cooling section 88. This applies in particular in the case of a plurality of conveying vehicles 18 positioned one behind the other within the drying device 84.

A drying section 86 can be arranged in particular above a nozzle device 90 and/or perforated plate 92 for the supply of a cooling air flow.

The drying device 84 can advantageously be formed for the convection drying of a screen-printed workpiece and/or for the blowing air cooling of workpiece carriers 20, in particular for the convection drying of a screen-printed workpiece with simultaneous blowing air cooling of the workpiece carrier 20 carrying the respective screen-printed workpiece.

A blowing air cooling can take place in this case via the nozzle device 90 and/or perforated plate 92 for the supply of a cooling air flow. A convection drying of screen-printed workpieces can take place via a drying air supply 94.

In particular, the drying device 84 can be formed to direct a drying air flow 95 onto a screen-printed workpiece or simultaneously onto a plurality of screen-printed workpieces, preferably at an angle to a transport axis formed by the transport rail 16 and/or at an angle relative to a transport straight line, by which a transport direction of the respective screen-printed workpiece and/or of the respective workpiece carrier 20 is defined, and/or transversely to the transport direction thereof and/or on the upper side onto the respective screen-printed workpiece.

If a drying air flow 95 is directed at an angle onto a screen-printed workpiece, this can be a direction of the drying air flow 95 which differs from a transport axis formed by the transport rail 16 or from the transport direction of the respective screen-printed workpiece and/or of the respective workpiece carrier 20. It can be a direction of the drying air flow 95 which is inclined with respect to the transport direction of the respective screen-printed workpiece and/or of the respective workpiece carrier 20, namely is inclined by a specific or predefined angle. A direction of the drying air flow 95 transversely to the transport direction of the respective screen-printed workpiece and/or of the respective workpiece carrier 20 runs in particular at a right angle or at an angle of 90° relative to a transport axis or relative to a transport straight line, by which a transport direction of the respective screen-printed workpiece and/or of the respective workpiece carrier 20 is defined.

An upper-side supply of a drying air flow 95 onto a screen-printed workpiece or onto a workpiece carrier 20 can be comprehended from the schematic illustrations in FIGS. 34 and 35. The downward-pointing arrow illustrations indicate an upper-side supply of a drying air flow 95 onto a screen-printed workpiece or onto a workpiece carrier 20.

Furthermore, the drying device 84 can be formed to extract air flows such that an extraction air flow 97 is established at an angle to a transport axis formed by the transport rail 16 and/or relative to a transport straight line, by which a transport direction of the respective screen-printed workpiece and/or of the respective workpiece carrier 20 is defined, in particular transversely to the transport axis and/or transport direction and/or in a horizontal direction and/or in a substantially horizontal direction from the respective screen-printed workpiece and/or workpiece carrier 20. Such an extraction of a drying air flow 95 can likewise be comprehended from the schematic illustrations in FIGS. 34 and 35. The sideways and upward-pointing arrow illustrations indicate such an extraction air flow 97 from a screen-printed workpiece or from a workpiece carrier 20.

Furthermore, the drying device 84 can be set up to direct extracted drying air and/or cooling air in the form of an extraction air flow 97 again as drying air onto a screen-printed workpiece to be dried, in particular as freshly supplied drying air flow 95.

Such an air guidance within the drying device 84 can be comprehended from the schematic illustrations in FIGS. 34 and 35 or the arrow illustrations contained therein. Consequently, the drying device 84 can be formed and/or configured as a circulating air dryer.

Likewise, the drying device 84 can be designed and/or configured for a mixed operation according to which a partial return of extracted drying air flows 95 and/or cooling air flows takes place. In a mixed operation, extracted drying air and/or cooling air can be mixed with fresh air after the return before said fresh air is directed again onto a screen-printed workpiece for the purpose of drying.

Likewise, the drying device 84 can be formed and/or configured with a heat exchanger, not illustrated in more detail here, for heat recovery. In such a configuration, the drying device 84 can direct exclusively fresh air onto the respective screen-printed workpieces to be dried and use heat energy recovered for heating the fresh air.

For heating a drying air flow 95 for the convection drying or for a drying air flow 95 to be directed through the drying air supply 94 onto the respective screen-printed workpieces, heating elements 96 can be provided, in particular above the heating air supply 94.

A drying air flow 95 directed past the heating elements 96 can therefore be suitably heated and then supplied via the drying air supply 94 to the respective screen-printed workpieces to be dried on the respective workpiece carrier 20.

The drying air supply 94 can advantageously have a plurality of nozzles 98, via which a drying air flow 95 can be directed onto the respective screen-printed workpieces for convection drying. The nozzles 98 can be designed in particular as slot nozzles.

A distance of the nozzles 98 relative to a conveying vehicle 18 which can be positioned in the drying device 84 can preferably be adjustable.

A distance of the nozzles 98 relative to a workpiece carrier 20 arranged or fixed in the conveying vehicle 18 can be adjustable by at least 30 mm, in particular by at least 35 mm, preferably by at least 40 mm or by at least 45 mm. During operation, this allows a relatively extensive adaptation of the distance of the nozzles 98 to a workpiece carrier 20 and therefore a large number of adjustment possibilities for different applications.

Further preferably, a distance of the nozzles 98 relative to a workpiece carrier 20 arranged or fixed in the conveying vehicle 18 can be adjustable by up to 90 mm, in particular by up to 80 mm, preferably by up to 70 mm or by up to 60 mm. This ensures an overall compact and robust construction despite the adjustability.

In a further preferred manner, a distance of the nozzles 98 relative to a workpiece carrier 20 arranged or fixed in the conveying vehicle 18 can be between 90 mm and 30 mm, in particular between 90 mm and 35 mm, in particular between 90 mm and 40 mm, preferably between 80 mm and 30 mm, preferably between 80 mm and 40 mm. Such an adjustability ensures a high number of different adjustment possibilities with a simultaneously robust and compact construction.

In a further preferred manner, the drying device 84 can be set up for the activation and/or deactivation of cooling positions 100 as a function of the position of a workpiece carrier 20 within the drying device 84, in particular for the activation of cooling positions 100 below the respective current position of a workpiece carrier 20.

In this case, a cooling position 100 can be set up for the activation by selective actuation of individual or a plurality of cooling nozzles 102 and/or compressed air openings 104 in and/or on a nozzle device 90 and/or perforated plate 92.

The cooling nozzles 102 and/or compressed air openings 104 can be formed as punctiform openings or also as longitudinally extended openings, in particular openings with a longitudinal extent at an angle to a transport axis formed by the transport rail 16, by which a transport direction of the conveying vehicle 18 is defined, and/or at an angle to the longitudinal extent of the transport rail 16. In particular, a longitudinally extended opening of a cooling nozzle 102 and/or compressed air opening 104 can have a longitudinal extent which encloses an angle, in particular encloses an angle of 90° or approximately 90°, with the transport axis and/or transport direction of the conveying vehicle 18 and/or with a longitudinal extent of the transport rail 16 in a plan view of the drying device 84.

In this case, a cooling position 100 can be formed by one or a plurality of cooling nozzles 102 and/or compressed air openings 104 which is/are arranged within the drying device 84 at a position along the transport rail 16 and/or along the transport direction of the conveying vehicle 18. The cooling nozzles 102 and/or compressed air openings 104 can be formed for example at regular intervals on a supply line 105, in particular on the upper side of a supply line 105. Likewise, a cooling position 100 can be formed by a longitudinally extended opening.

A plurality of cooling nozzles 102 and/or compressed air openings 104 of a cooling position can be arranged distributed at an angle or transversely to a transport axis formed by the transport rail 16 or transversely to the transport direction of the conveying vehicle 18 and/or at an angle to the longitudinal extent of the transport rail 16. In particular, a plurality of cooling nozzles 102 and/or compressed air openings 104 of a cooling position can be arranged distributed along a direction which encloses an angle, in particular encloses an angle of 90° or approximately 90°, with the transport direction of the conveying vehicle 18 and/or with a longitudinal extent of the transport rail 16 in a plan view of the drying device 84.

A cooling nozzle 102 and/or compressed air opening 104 of a cooling position formed as a longitudinally extended opening can extend at an angle or transversely to a transport axis formed by the transport rail 16 or transversely to the transport direction of the conveying vehicle 18 and/or at an angle to the longitudinal extent of the transport rail 16. In particular, a cooling nozzle 102 and/or compressed air opening 104 of a cooling position formed as a longitudinally extended opening can extend along a direction which encloses an angle, in particular encloses an angle of 90° or approximately 90°, with a transport axis formed by the transport rail 16 or with the transport direction of the conveying vehicle 18 and/or with a longitudinal extent of the transport rail 16 in a plan view of the drying device 84.

In a still further preferred manner, the drying device 84 can have at least one air filter 106 for the filtering of a drying air stream 95. The air filter 106 can be designed in particular as a HEPA air filter. The nozzles 98 of the drying air supply 94 can therefore be provided and/or arranged for the passage or supply of filtered drying air or of an already filtered drying air flow 95.

Finally, the drying device 84 can also be formed for the continuous and/or periodic supply of fresh air and/or room air.

When a conveying vehicle 18 is positioned within the drying device 84, the nozzle device 90 and/or perforated plate 92 can project at least in sections into the free space 44 of the superstructure 36 for receiving a printing table and/or between the base section 46 and the carrying section 40 of the superstructure 36. In such a position, the nozzle device 90 and/or perforated plate 92 can project at least in sections below a workpiece carrier 20 received by the respective conveying vehicle 18. In this way, a direct supply of a cooling air flow to the underside of the respective workpiece carrier 20 can take place and a suitable cooling can therefore be ensured.

In a position projecting at least in sections into the free space 44 of the superstructure 36 for receiving a printing table, the nozzle device 90 and/or perforated plate 92 can further preferably serve as an insulating device between different sections of the drying device 84. In particular, the nozzle device 90 and/or perforated plate 92 can serve as an insulating device between a drying section 86 and a cooling section 88. For this purpose, the nozzle device 90 and/or perforated plate 92 can extend along a horizontal plane or substantially along a horizontal plane.

The nozzle device 90 and/or perforated plate 92 can in this case itself be part of a cooling section 88 of the drying device 84, but promote an insulation between a drying section 86 and a cooling section 88. This applies in particular to a position in which the nozzle device 90 and/or perforated plate 92 projects into the free space 44 of the superstructure 36 for receiving a printing table. In this case, the nozzle device 90 and/or perforated plate 92 can namely direct cooling air only onto the underside of a workpiece carrier 20 received in the respective superstructure 36 and at least temporarily avoid an undesired mixing with drying air directed onto the respective workpiece carrier 20 on the upper side or at least reduce such a mixing.

Furthermore, by means of a nozzle device 90 and/or perforated plate 92, it can be avoided that a warm drying air flow reaches regions below the nozzle device 90 and/or perforated plate 92 and brings about an undesired heating there. Overall, the nozzle device 90 and/or perforated plate 92 can provide an advantageous insulating function.

The drying device 84 can have an opening 108 on at least one longitudinal end, which opening is formed at least in sections in a complementary manner, in particular in a geometrically complementary manner, to the cross-sectional shape and/or in a complementary manner, in particular in a geometrically complementary manner, to a cross-sectional sectional shape of the conveying vehicle 18. The escape of air quantities from the drying device 84 can be reduced to a low degree as a result.

The apparatus 10 can furthermore be equipped with a positioning and/or removal apparatus 110 for the positioning of workpiece carriers 20 on a conveying vehicle 18 and/or for the removal of a workpiece carrier 20 from a conveying vehicle 18.

Such a positioning and/or removal apparatus 110 is illustrated schematically in FIGS. 40 to 43.

A positioning and/or removal apparatus 110 can be formed in particular to actuate a slide 70 of the locking mechanism 68 in order thereby to carry out an unlocking of a workpiece carrier 20 received in a conveying vehicle 18. An actuation of the slide 70 can preferably take place in an automated manner by the positioning and/or removal apparatus 110, in particular by a claw 112 for engaging behind the slide 70.

Furthermore, the positioning and/or removal apparatus 110 can be formed to carry out an automated handling of a workpiece carrier 20 for the purpose of removal from the conveying vehicle 20 after unlocking of the locking mechanism 68 and/or for the purpose of positioning within the conveying vehicle 18 after opening of the slide 70. For this purpose, for example, a pressing apparatus 114 can be provided, by means of which a workpiece carrier 20 can be raised out of the conveying vehicle 18 at least on one side or can be transferred into an inclined position. A manual removal of the workpiece carrier 20 or else a further automated removal can take place from such an inclined position of the workpiece carrier 20.

After a different or new workpiece carrier 20 has been arranged in the respective conveying vehicle 18, a pressing apparatus 114 can be lowered again, with the result that the workpiece carrier 20 is located in a planar position lying on the conveying vehicle 18. Subsequently, the positioning and/or removal apparatus 110, in particular the claw 112, can release the slide 70 of the locking mechanism 68 and/or guide it into a locking position in a controlled manner in order to bring about a fixing of the respective workpiece carrier 20.

Furthermore, a positioning and/or removal apparatus 110 can be equipped with a detection apparatus 116, by means of which workpiece carriers 20 to be removed and/or newly positioned on the respective conveying vehicle 18 can be detected. An apparatus 10 described above is suitable in a particularly preferred manner for carrying out a method for producing three-dimensional screen-printed workpieces. In such a method, at least one screen-printed workpiece is produced layerwise by the printing device 12 in a plurality of printing operations. At least one screen-printed workpiece and/or a workpiece carrier 20 with a screen-printed workpiece is transported in an automated manner towards and/or away from the printing device 12 by the transport device 14, wherein the transport by the transport device 14 can be carried out by means of the conveying vehicle 18 on the transport rail 16.

The apparatus 10 may in particular be designed and/or set up for the development and/or for the production of large quantities of pharmaceutical products.

### LIST OF REFERENCE SIGNS

10 Apparatus for producing three-dimensional screen-printed workpieces
12 printing device
14 transport device
16 transport rail
18 conveyor vehicle
20 workpiece carrier
22 housing
24 printing table
26 drive device
28 drive roller
29 current-carrying contact roller
30 supporting device
32 supporting roller
33 control module
34 apparatus controller
35 control unit
36 superstructure
38 runner device
40 carrying section
42 side end
44 free space
46 base section
48 connecting section
50 stroke-limiting device
52 stroke-limiting claw
54 multi-point bearing
56 bearing
58 bearing mandrel
60 mandrel receptacle
61 adjusting screw
73
62 frame device
63 thread
64 workpiece carrier mounting
66 fixed mounting
68 locking mechanism
70 slide
72 contact surface
74 contact surface
76 support section
78 shaped element
80 bypass section
82 main section
84 drying device
86 drying section
88 cooling section
90 nozzle device
92 perforated plate
94 drying air supply
95 drying air flow
96 heating element
97 extraction air flow
98 nozzle
100 cooling position
102 cooling nozzle
104 compressed air opening
106 air filter
108 opening on longitudinal end of the drying device
110 positioning and/or removal apparatus

1. The apparatus (10) for producing three-dimensional screen-printed workpieces, comprising a printing device (12) for the layer-wise production of at least one screen-printed workpiece in a plurality of printing operations; and a transport device (14) for the automated transport of the at least one screen-printed workpiece and/or of a workpiece carrier (20) towards and/or away from the printing device (12), wherein the transport device (14) has a carrying section (40) for at least form-fitting reception of the workpiece carrier (20).

2. The apparatus (10) according to claim 1, wherein the transport device (14) is set up for the automated transport of the at least one screen-printed workpiece and/or of the workpiece carrier (20) towards and/or away from a printing table (24) of the printing device (12), and/or wherein the transport device (14) is formed separately from the printing device (12), and/or wherein the transport device (14) runs through the printing device (12).

3. The apparatus (10) according to claim 1, wherein the transport device (14) has at least one transport rail (16) and a conveying vehicle (18), arranged movably on the transport rail (16), for the at least one screen-printed workpiece and/or for the workpiece carrier (20), wherein the carrying section (40) for receiving the workpiece carrier (20) is arranged on the conveying vehicle (18).

4. The apparatus (10) according to claim 1, wherein the carrying section (40) is designed for receiving and/or fixing the workpiece carrier (20) in a form-fitting and/or force-fitting and/or material-bonded manner and/or at least in sections and/or wherein the carrying section (40) is designed for receiving and/or enclosing the workpiece carrier (20) in a defined and/or play-free manner.

5. The apparatus (10) according to claim 1, wherein the carrying section (40) has a frame device (62), designed for receiving and/or enclosing the workpiece carrier (20) in a defined and/or play-free manner; optionally wherein the frame device (62) delimits a free space (44) for contacting the underside of the workpiece carrier (20) received and/or enclosed therein.

6. The apparatus (10) according to claim 1, wherein the transport device (14) has a workpiece carrier mounting (64) for fixing and/or for clamping and/or for fastening the workpiece carrier (20) in a force-fitting and/or form-fitting manner, wherein the workpiece carrier mounting (64) is designed as a multi-point bearing.

7. The apparatus (10) according to claim 6, wherein the workpiece carrier mounting (64) is formed by at least one fixed mounting (66) and/or by at least one locking mechanism (68).

8. The apparatus (10) according to claim 7, wherein the locking mechanism (68) is formed by a slide (70) with a contact surface (72) which is chamfered and/or inclined with respect to the sliding direction of the slide (70), and/or wherein the locking mechanism (68) is formed by a slide (70) which is under spring pretension and/or as a snap closure.

9. The apparatus (10) according to claim 7, wherein at least one fixed mounting (66) is formed with a contact surface (74) which is chamfered and/or inclined with respect to the sliding direction of the slide (70).

10. (canceled)

11. The apparatus (10) according to claim 3, wherein the conveying vehicle (18) has a superstructure (36) for receiving the workpiece carrier (20) for the at least one screen-printed workpieces and/or a runner device (38) with a drive device (26) arranged thereon, wherein the superstructure (36) is arranged on the runner device (38) of the conveying vehicle (18) and/or wherein the carrying section (40) is formed on the superstructure (36).

12. (canceled)

13. (canceled)

14. The apparatus (10) according to claim 11, wherein the superstructure (36) is C-shaped and/or forms a free space (44) for receiving a printing table (24) of the printing device (12) and/or wherein, in a cross-section of the superstructure (36), the surface center is arranged laterally offset with respect to the center of gravity of the superstructure (36).

15. (canceled)

16. The apparatus (10) according to claim 11, wherein the superstructure (36) is mounted movably relative to the runner device (38) in the vertical direction and/or is arranged so as to be adjustable in height and/or wherein the superstructure (36) is arranged so as to be raisable relative to the runner device (38) by a printing table (24) of the printing device (12).

17. (canceled)

18. (canceled)

19. The apparatus (10) according to claim 11, wherein the superstructure (36) is fastened on the runner device (38) and/or guided in a vertically movable manner via a multi-point bearing (54) and/or wherein at least one bearing (56) between the superstructure (36) and the runner device (38) is formed by a bearing mandrel (58) and a mandrel receptacle (60) for the bearing mandrel (58).

20. The apparatus (10) according to claim 19, wherein the multi-point bearing (54) and/or at least one bearing (56) between the superstructure (36) and the runner device (38) is designed for self-centering and/or self-aligning the superstructure (36) relative to the runner device (38) and/or wherein at least one bearing mandrel (58) tapers conically and/or in that at least one mandrel receptacle (60) tapers conically.

21-25. (canceled)

26. The apparatus (10) according to one claim 11, wherein the superstructure (36) has supporting sections (76) for contacting and/or raising the superstructure by a printing table (24) of the printing device (12), wherein the supporting sections (76) are formed on an underside of the carrying section (40) and/or of a frame device (62).

27. (canceled)

28. The apparatus (10) according to claim 1, wherein the printing device (12) has a liftable printing table (24) and/or in that wherein the printing table (24) of the printing device (12) is designed for planar contacting of the workpiece carrier (20) received in a conveying vehicle (18) and/or for contacting the supporting sections (76) of the superstructure (36) of the conveying vehicle (18).

29-48. (canceled)

49. A transport device for the transport in an apparatus (10) for producing three-dimensional screen-printed workpieces, comprising: a conveying vehicle (18) for the automated transport of at least one screen-printed workpiece and/or of a workpiece carrier (20), wherein the conveying vehicle (18) has a carrying section (40) for at least form-fitting reception of the workpiece carrier (20) and/or a workpiece carrier mounting for the fixing and/or for the clamping of the workpiece carrier (20).

50. A method for producing three-dimensional screen-printed workpieces, using the apparatus (10) according to claim 1, in which the at least one screen-printed workpiece is produced layer-wise in a plurality of printing operations by the printing device (12) and in which the at least one screen-printed workpiece on the workpiece carrier (20) is transported in an automated manner towards and/or away from the printing device (12) by the transport device (14), wherein the workpiece carrier (20) is received at least in a form-fitting manner on and/or in a carrying section (40) for transport by the transport device (14).

51. A method for producing three-dimensional screen-printed workpieces using the apparatus (10) according to claim 1, in which the at least one screen-printed workpiece is produced layer-wise in a plurality of printing operations by the printing device (12) and in which the at least one screen-printed workpiece on the workpiece carrier (20) is transported in an automated manner towards and/or away from the printing device (12) by a transport device (14), wherein the workpiece carrier (20) is fixed and/or clamped in a workpiece carrier mounting (64) for the transport by the transport device (14).

52. The apparatus (10) according to claim 11, wherein the printing device (12) has a liftable printing table (24) designed to raise the superstructure (36) via the supporting sections (76) in a manner load-free for the workpiece carrier mounting (64).
